(19) United States
(12) Patent Application Publication
(10) Pub. No.: US 2025/0195218 A1
(43) Pub. Date: Jun. 19, 2025

(54) DELIVERY APPARATUS FOR A PROSTHETIC DEVICE

(71) Applicant: EDWARDS LIFESCIENCES CORPORATION, Irvine, CA (US)

(72) Inventor: Peleg Harel, Haifa (IL)

(73) Assignee: EDWARDS LIFESCIENCES CORPORATION, Irvine, CA (US)

(21) Appl. No.: 19/069,423

(22) Filed: Mar. 4, 2025

Related U.S. Application Data
(63) Continuation of application No. PCT/US2023/032108, filed on Sep. 6, 2023.
(60) Provisional application No. 63/404,496, filed on Sep. 7, 2022.

(51) Int. Cl. A61F 2/24

(52) U.S. Cl. CPC A61F 2/2427

(57) ABSTRACT

A delivery apparatus for a prosthetic valve comprises a handle body, and shaft displacement and shaft adjustment mechanisms coupled to the handle body. The shaft displacement mechanism is configured to axially displace a shaft relative to the handle body. The adjustment mechanism is configured to adjust a curvature of the shaft and comprises a pull wire coupled to a distal end of the shaft. A first knob is operatively coupled to the shaft displacement mechanism and rotatable relative to the handle body, wherein rotating the first knob relative to the handle body simultaneously axially displaces the shaft and the pull wire relative to the handle body. A second knob is operatively coupled to the shaft adjustment mechanism and rotatable relative to the handle body, wherein rotating the second knob relative to the handle body adjusts the curvature of the shaft independent of an axial displacement of the shaft.

### CROSS REFERENCE TO RELATED APPLICATIONS

This application is a continuation of PCT Application No. PCT/US2023/032108, filed Sep. 6, 2023, which claims the benefit of U.S. Provisional Application No. 63/404,496, filed Sep. 7, 2022, both of which are incorporated by reference herein.

### FIELD

The present disclosure relates to apparatus and methods for delivering, expanding, and implanting implantable, radially expandable prosthetic devices, such as prosthetic heart valves, stents, or the like.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Described herein are prosthetic heart valves, delivery apparatus, and methods for implanting prosthetic heart valves. The disclosed prosthetic heart valves, delivery apparatus, and methods can, for example, provide for manipulation of a radius of curvature of a shaft of a delivery apparatus independent of an axial displacement of the shaft relative to other components of the delivery apparatus and/or relative to a prosthetic device. As such, the devices and methods disclosed herein can, among other things, overcome one or more of the deficiencies of typical prosthetic heart valves and their delivery apparatus.

A delivery apparatus for a prosthetic implant can comprise a handle and a shaft coupled to the handle.

In some examples, a delivery apparatus can comprise a shaft displacement mechanism coupled to the handle and a first knob operatively coupled to the shaft displacement mechanism and rotatable relative to the handle.

In some examples, a delivery apparatus can comprise shaft adjustment mechanism coupled to the handle and a second knob operatively coupled to the shaft adjustment mechanism and rotatable relative to the handle.

In some examples the shaft adjustment mechanism comprises a pull wire coupled to a distal end of the shaft.

In some examples, the shaft displacement mechanism can be configured such that rotating the first knob relative to the handle simultaneously axially displaces the shaft and the pull wire relative to the handle.

In some examples, the shaft adjustment mechanism can be configured such that rotating the second knob relative to the handle adjusts the curvature of the shaft independent of an axial displacement of the shaft.

In some examples, the shaft adjustment mechanism can include a rotatable, adjustment barrel having a lumen that includes a threaded inner surface, and an adjustment nut coupled to the pull wire and disposed within the lumen.

In some examples, the adjustment nut can include a threaded outer surface coupled to the threaded inner surface of the adjustment barrel, and the adjustment nut can be configured to translate axially relative to the adjustment barrel in response to rotation of the adjustment barrel.

In some examples, the adjustment nut can an attachment member extending radially from a main body of the adjustment nut, and the proximal end of the pull wire is wrapped around the attachment member.

In some examples, the delivery apparatus can include a connector shaft coupled to the adjustment mechanism and the shaft displacement mechanism.

In some examples, the adjustment nut can be disposed circumferentially around the connector shaft and can be movable axially relative to the connector shaft.

In some examples, the delivery apparatus can include a gear system operatively coupling the shaft adjustment mechanism and the second knob.

In some examples, the delivery apparatus can include a gear system operatively coupling the shaft displacement mechanism and the first knob.

In some examples, the delivery apparatus can include a rotatable indicator coupled to the shaft adjustment mechanism and configured to indicate the curvature of the shaft upon rotation of the second knob.

In some examples, the shaft displacement mechanism can comprise a displacement nut coupled to the shaft.

In some examples, the displacement nut can be threadedly coupled to the first knob, such that rotation of the first knob relative to the handle results in axial displacement of the displacement nut and the shaft relative to the handle.

In some examples, the displacement nut can be threadedly coupled to one or more threaded rods, each of the one or more threaded rods coupled to a gear meshed with an interior surface of the first knob, wherein rotation of the first knob relative to the handle results in axial displacement of the displacement nut and the shaft relative to the handle.

In some examples, the first knob can be proximal of the second knob and the displacement nut, such that the first knob and the second knob are axially separated on the handle of the delivery apparatus.

In some examples, a delivery apparatus comprises one or more of the components recited in Examples 1-95 and 103-106 below.

A prosthetic heart valve for use with the delivery apparatus disclosed herein can comprise a frame and a valve structure coupled to the frame. In addition to these components, a prosthetic heart valve can further comprise one or more of the components disclosed herein.

A method of using a delivery apparatus for delivery of a prosthetic implant can comprise adjusting a curvature of a delivery shaft that retains the prosthetic implant relative to a longitudinal axis of a handle coupled to the delivery shaft.

In some examples, the method can include displacing the delivery shaft relative to the prosthetic implant, wherein the curvature is maintained during displacement.

In some examples, adjusting the curvature can comprise rotating a first knob relative to the handle.

In some examples, displacing the delivery shaft relative to the prosthetic implant can comprise rotating a second knob relative to the handle.

In some examples, a method comprises one or more of the features recited in Examples 96-102 below.

The above method(s) can be performed on a living animal or on a simulation, such as on a cadaver, cadaver heart, anthropomorphic ghost, simulator (e.g., with body parts, heart, tissue, etc. being simulated).

In some examples, a delivery apparatus for a prosthetic valve comprises a handle body; a shaft displacement mechanism coupled to the handle body, the shaft displacement mechanism configured to axially displace a shaft relative to the handle body; a shaft adjustment mechanism coupled to the handle body, the adjustment mechanism configured to adjust a curvature of the shaft, the shaft adjustment mechanism comprising a pull wire coupled to a distal end of the shaft; a first knob operatively coupled to the shaft displacement mechanism and rotatable relative to the handle body, wherein rotating the first knob relative to the handle body simultaneously axially displaces the shaft and the pull wire relative to the handle body; and a second knob operatively coupled to the shaft adjustment mechanism and rotatable relative to the handle body, wherein rotating the second knob relative to the handle body adjusts the curvature of the shaft independent of an axial displacement of the shaft.

In some examples, a handle for a delivery apparatus for a prosthetic valve comprises a handle body; a shaft displacement mechanism coupled to the handle body, the shaft displacement mechanism configured to axially displace a shaft relative to the handle body; a shaft adjustment mechanism coupled to the handle body, the adjustment mechanism configured to adjust a curvature of the shaft, the shaft adjustment mechanism comprising a pull wire coupled to a distal end of the shaft; a first knob operatively coupled to the shaft displacement mechanism and rotatable relative to the handle body, wherein rotating the first knob relative to the handle body simultaneously axially displaces the shaft and the pull wire relative to the handle body; and a second knob operatively coupled to the shaft adjustment mechanism and rotatable relative to the handle body, wherein rotating the second knob relative to the handle body adjusts the curvature of the shaft independent of an axial displacement of the shaft.

In some examples, a delivery apparatus for a prosthetic valve comprises a delivery shaft; at least one expansion mechanism disposed within the delivery shaft; a displacement nut coupled to a proximal end portion of the delivery shaft, the displacement nut configured to axially displace the delivery shaft relative to the expansion mechanism; a shaft adjustment mechanism comprising a pull wire coupled to a distal end of the delivery shaft, the adjustment mechanism configured to adjust a curvature of the delivery shaft; a connector shaft coupled to the displacement nut and the shaft adjustment mechanism; a first knob operatively coupled to the displacement nut and rotatable relative to the expansion mechanism, wherein rotating the first knob relative to the expansion mechanism simultaneously axially displaces the displacement nut, the delivery shaft, the pull wire, and the connector shaft relative to the expansion mechanism; and a second knob operatively coupled to the shaft adjustment mechanism and rotatable relative to the expansion mechanism, wherein rotating the second knob relative to the expansion mechanism adjusts a tension of the pull wire independent of an axial displacement of the delivery shaft.

In some examples, a delivery apparatus for a prosthetic valve comprises a delivery shaft; at least one expansion mechanism disposed within the delivery shaft; a displacement member coupled to a proximal end portion of the delivery shaft, the displacement member configured to axially displace the delivery shaft relative to the expansion mechanism; a pull wire coupled to a distal end of the delivery shaft, the pull wire configured to adjust a curvature of the delivery shaft; an adjustment nut coupled to the pull wire, wherein the adjustment nut includes a threaded outer surface; and a rotatable, adjustment barrel having a threaded inner surface coupled to the threaded outer surface of the adjustment nut, wherein rotation of the adjustment barrel relative to the expansion member results in axial displacement of the adjustment nut relative to the adjustment barrel.

In some examples, a method of implanting a prosthetic implant comprises adjusting a curvature of a delivery shaft that retains the prosthetic implant relative to a longitudinal axis of a handle coupled to the delivery shaft; and displacing the delivery shaft relative to the prosthetic implant, wherein the curvature is maintained during displacement.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2A is a perspective view of a frame for the prosthetic valve of FIG. 1A.

FIG. 2B is a front portion of the frame shown in FIG. 2A.

FIG. 3 is a side elevation view of a delivery apparatus for a prosthetic device, such as a prosthetic valve, according to one example.

FIG. 4 is a perspective view of a portion of an actuator of the prosthetic device of FIGS. 1-2 and an actuator assembly of a delivery apparatus, according to one example.

FIG. 5 is a perspective view of the actuator and actuator assembly of FIG. 4 with the actuator assembly physically coupled to the actuator.

FIG. 6 is a side elevation view of a delivery apparatus for a prosthetic device, such as a prosthetic valve, according to one example.

FIG. 7 is a partial cross-sectional view of the delivery apparatus of FIG. 6.

FIG. 8 is a perspective view of an adjustment member and connector shaft of the delivery apparatus of FIG. 6.

FIG. 9 is a detailed view of a cross-sectional view of the delivery apparatus of FIG. 6.

FIG. 10 is a section view of the delivery apparatus of FIG. 6, taken along section 10-10 (FIG. 7).

FIG. 11 is a cross-sectional view of an adjustment lead member meshed with gears of the delivery apparatus of FIG. 6.

FIG. 12 is a cross-sectional view of a distal end of the handle of the delivery apparatus of FIG. 6.

FIG. 13 is a cross-sectional view of a knob meshed with gears of the delivery apparatus of FIG. 6.

FIG. 14A is a cross-sectional view of the delivery apparatus of FIG. 6 with a displacement mechanism in a first position.

FIG. 14B is a cross-sectional view of the delivery apparatus of FIG. 6 with the displacement mechanism in a second position.

FIG. 15A is a cross-sectional view of a distal end of the delivery apparatus of FIG. 6 positioned within a heart, with the displacement mechanism in the first position of FIG. 14A.

FIG. 15B is a cross-sectional view of the distal end of the delivery apparatus of FIG. 6 positioned within the heart with the displacement mechanism in the second position of FIG. 14B.

FIG. 16 is a cross-sectional view of the delivery apparatus of FIG. 6 with the displacement mechanism in a third position and an adjustment mechanism in a first position.

FIG. 17 is a cross-sectional view of the delivery apparatus of FIG. 6 with the displacement mechanism in the third position and the adjustment mechanism in a second position.

FIG. 18 is a cross-sectional view of the distal end of the handle of the delivery apparatus of FIG. 6 with an end cap removed for illustration purposes.

FIG. 19 is a perspective view of the distal end of the handle of the delivery apparatus of FIG. 18.

FIG. 20 is a front elevation view of the distal end of the handle of the delivery apparatus of FIG. 18.

FIG. 21 is a side elevation view of the distal end of the handle of the delivery apparatus of FIG. 6.

FIG. 22 is a perspective view of a delivery apparatus for a prosthetic device, such as a prosthetic valve, according to another example.

FIG. 23 is a perspective view of the delivery apparatus of FIG. 22 with a housing of the delivery apparatus removed.

FIG. 24 is a cross-sectional view of the delivery apparatus of FIG. 22.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Overview of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

Described herein are examples of a steerable delivery apparatus (sometimes referred to as a steerable catheter) that can be used to navigate a subject's vasculature to deliver an implantable, expandable medical device (for example, a prosthetic heart valve), tools, agents, or other therapy to a location within the body of a subject. Examples of procedures in which the steerable catheters are useful include neurological, urological, gynecological, fertility (for example, in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

Delivery apparatuses described herein can include mechanisms to adjust (for example, control, steer, flex, etc.) a distal end portion of a shaft that retains an implantable, expandable medical device (for example, a prosthetic heart valve). The delivery apparatuses can also include mechanisms to displace (for example, advance, retract, etc.) the shaft in an axial direction relative to the medical device to deploy the medical device from the shaft at the implantation site. The shaft displacement mechanisms and the shaft adjustment mechanisms can be operated independently of each other, thus allowing greater manipulation of the distal end portion of the shaft. For example, the displacement mechanism can be utilized to retract the shaft to deploy the medical device, without affecting the curvature of the shaft during deployment. Additionally, the control mechanism can be utilized to flex the shaft without affecting the axial position of the shaft relative to a handle of the delivery apparatus. Delivery apparatuses described herein can also include mechanisms to indicate an amount of adjustment (for example, radius of curvature, etc.) of the shaft based on operation of the adjustment mechanism.

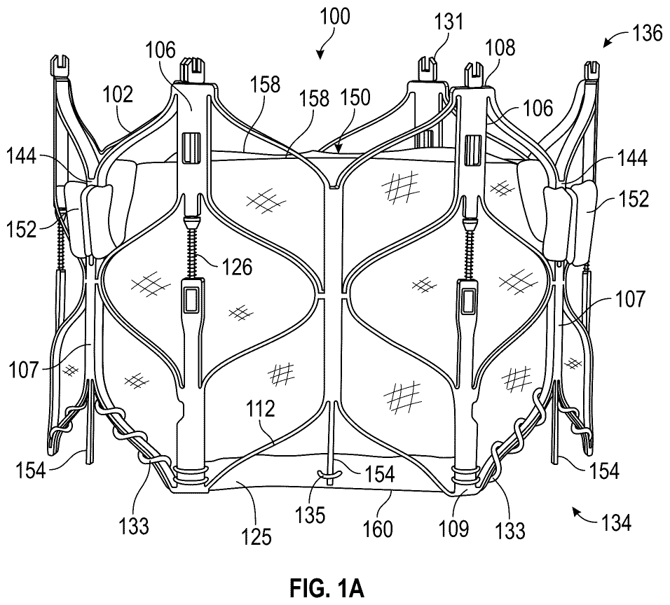

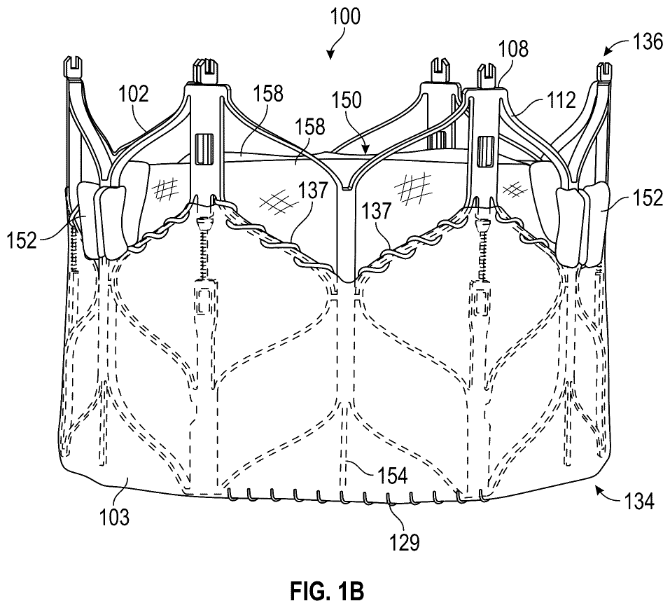

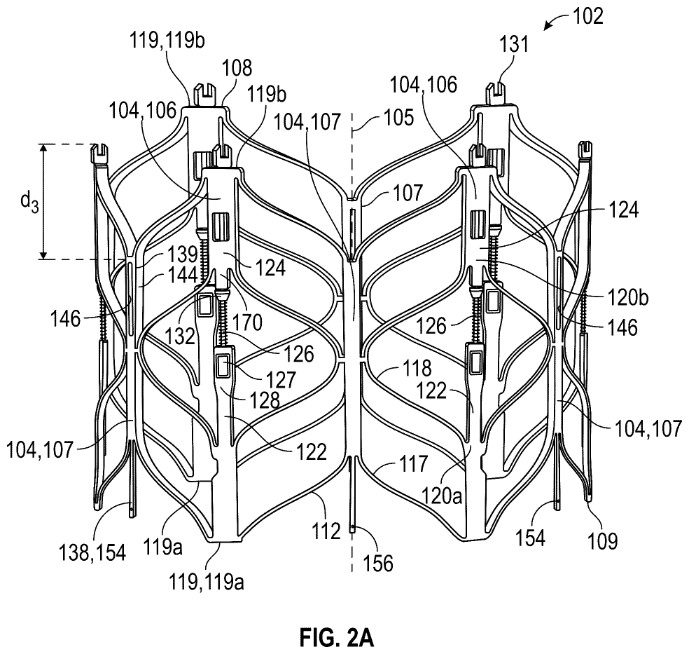

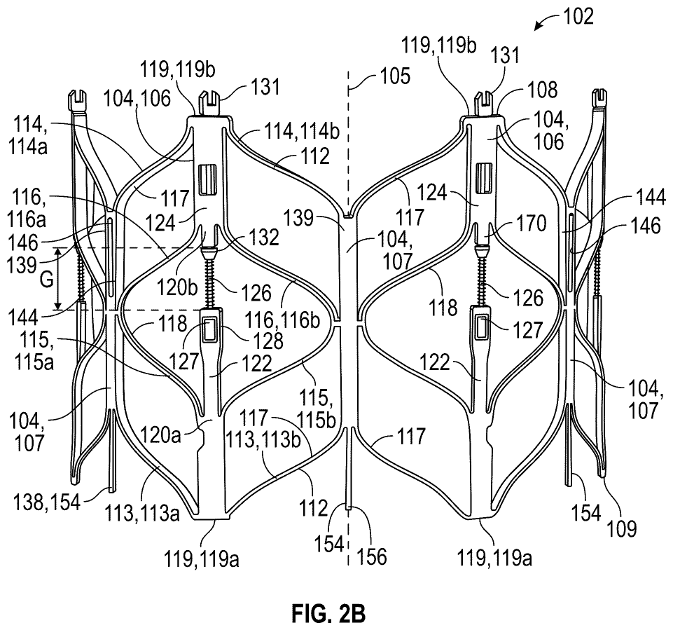

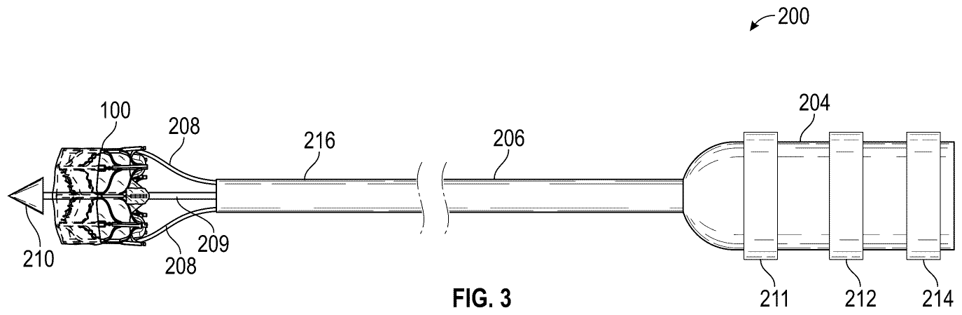

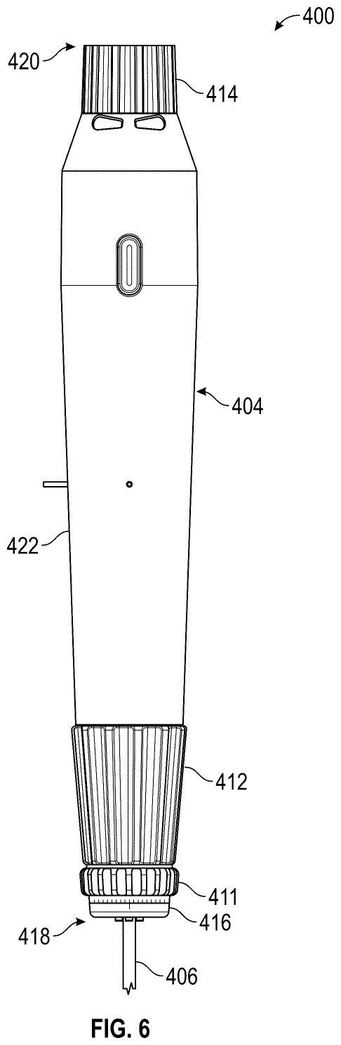

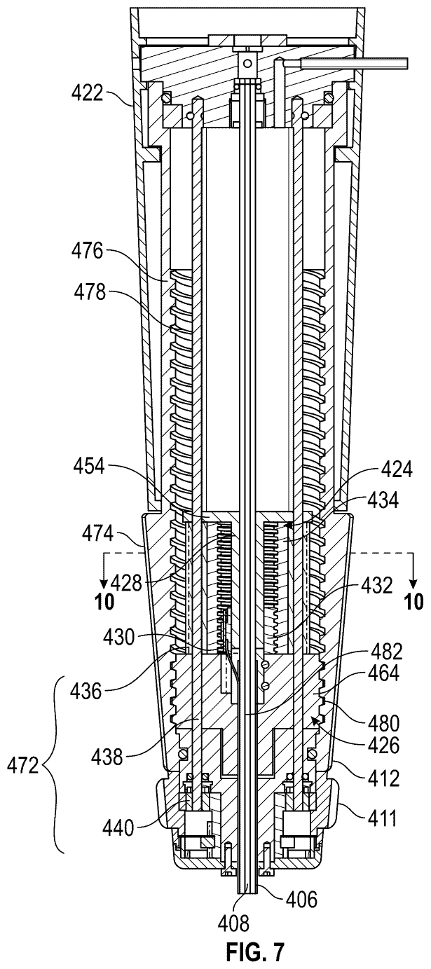

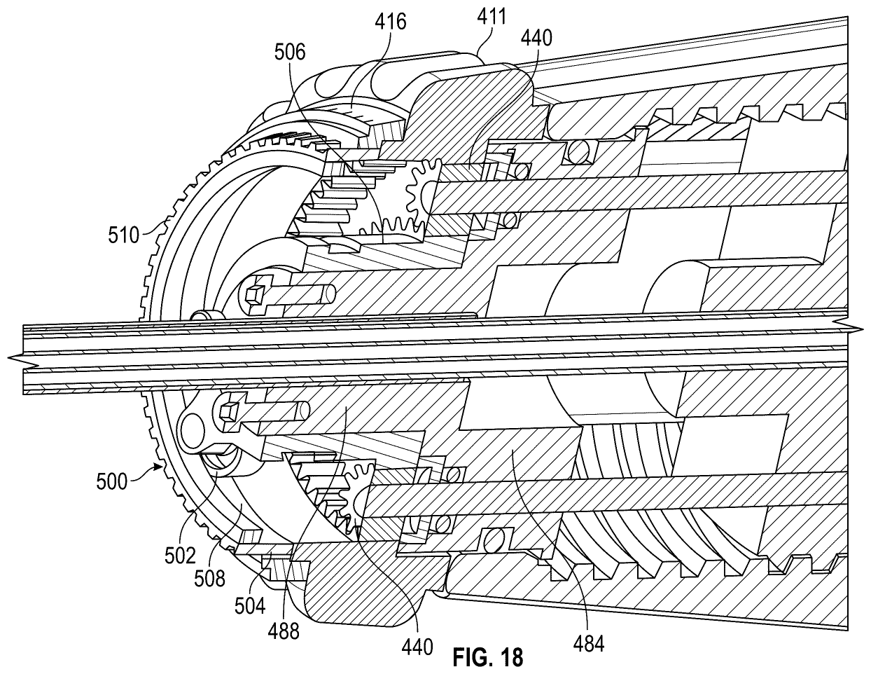

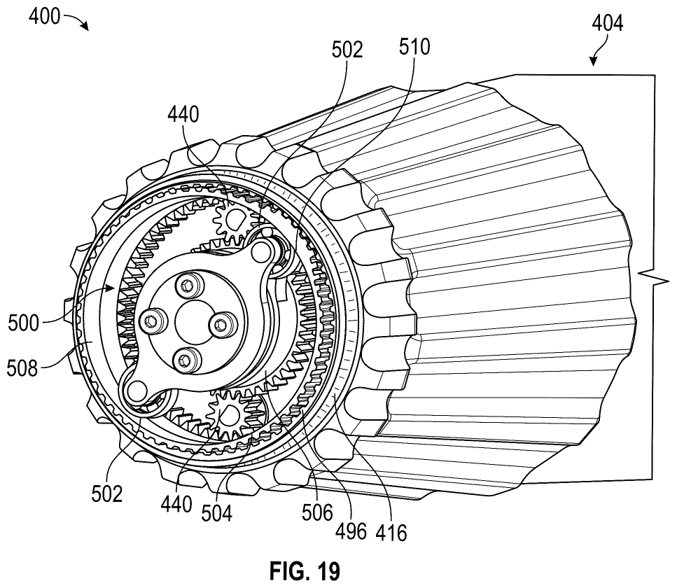

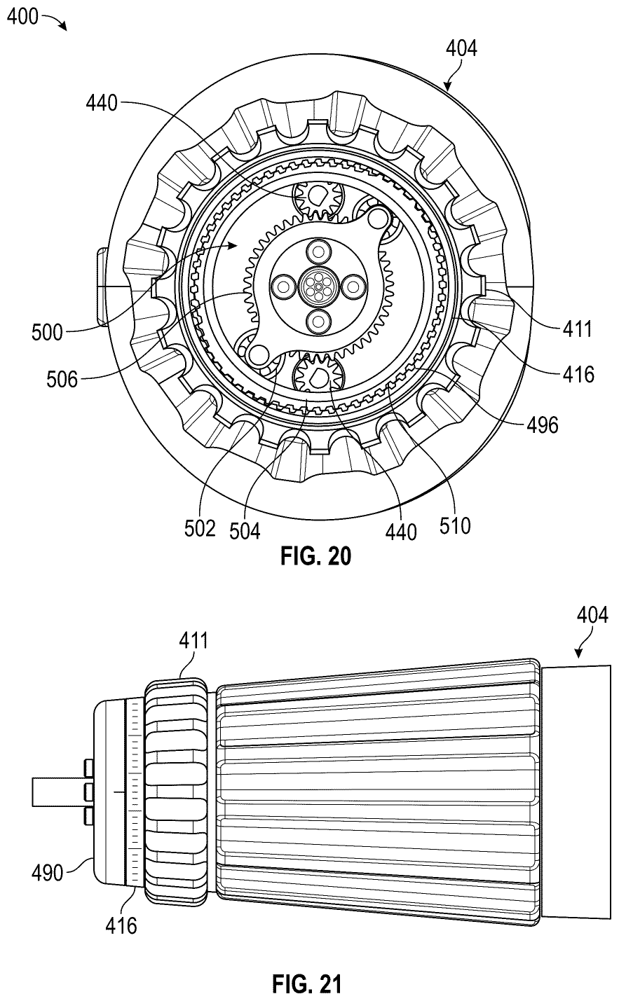

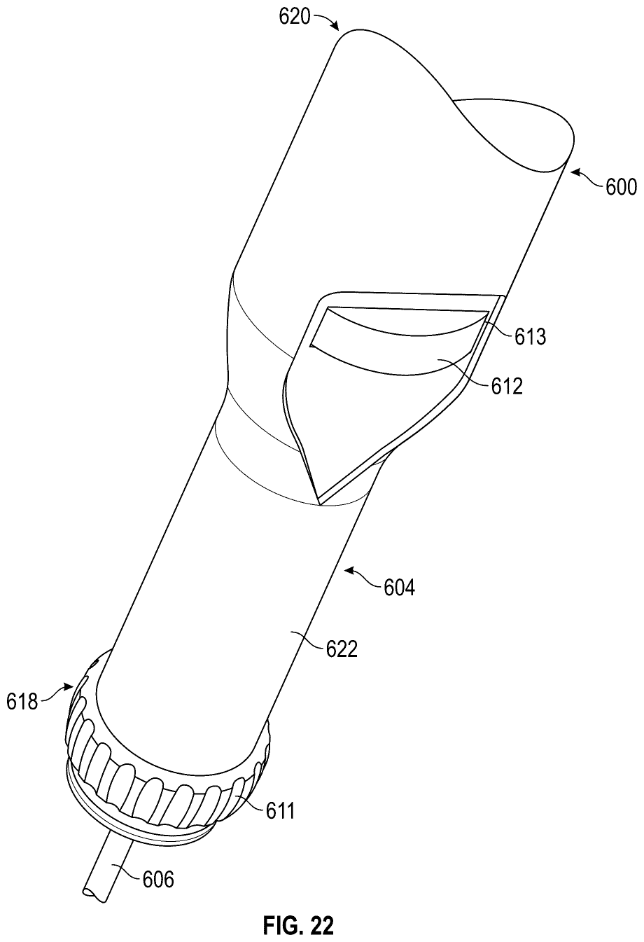

FIGS. 1A-2B illustrate an example of a medical device (for example, prosthetic heart valve) that can be advanced through a patient's vasculature, such as to a native heart valve, by a delivery apparatus, such as the delivery apparatus shown in FIG. 3, or the delivery apparatus shown in FIG. 6. Additional details of a shaft displacement mechanism and a shaft adjustment mechanism for a delivery apparatus are illustrated in FIGS. 7-17. An indicator mechanism for a delivery apparatus is illustrated in FIGS. 18-21. Another exemplary delivery apparatus in accordance with the present disclosure is illustrated in FIGS. 22-24.

### Examples of the Disclosed Technology

FIGS. 1A-2B show a prosthetic valve 100, according to one example. The prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries, and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIGS. 1A-2B illustrate an example of a prosthetic valve 100 (which also may be referred to herein as “prosthetic heart valve 100”) having a frame 102. FIGS. 2A-2B show the frame 102 by itself, while FIGS. 1A-1B show the frame 102 with a valvular structure 150 (which can comprise leaflets 158, as described further below) mounted within and to the annular frame 102. FIG. 1B additionally shows an optional skirt assembly comprising an outer skirt 103. While only one side of the frame 102 is depicted in FIG. 2B, it should be appreciated that the frame 102 forms an annular structure having an opposite side that is substantially identical to the portion shown in FIG. 1B, as shown in FIGS. 1A-2A.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail below.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of frame 102 in a circumferential direction when the frame 102 is in the radially expanded configuration. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 mounted around an outer surface of the frame 102. The outer skirt 103 can function as a sealing member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In some examples, as shown in FIG. 1A, the leaflets 158 can be sutured directly to the frame 102 or to a reinforcing member 125 (also referred to as a reinforcing skirt or connecting skirt) in the form of a strip of material (for example, a fabric strip) which is then sutured to the frame 102, along the scallop line via stitches (for example, whip stitches) 133.

The inner and outer skirts and the connecting skirt 125 can be formed from any of various suitable biocompatible materials, including any of various synthetic materials, including fabrics (for example, polyethylene terephthalate fabric) or natural tissue (for example, pericardial tissue). Further details regarding the use of skirts or sealing members in prosthetic valve can be found, for example, in U.S. Patent Publication No. 2020/0352711, which is incorporated herein by reference.

Further details regarding the assembly of the leaflet assembly and the assembly of the leaflets and the skirts to the frame can be found, for example, in International Patent Application No. PCT/US2022/032983, filed Jun. 10, 2022, and U.S. Provisional Application 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

The frame 102, which is shown alone and in greater detail in FIGS. 2A and 2B, comprises and inflow end 109, an outflow end 108, and a plurality of axially extending posts 104. The axial direction of the frame 102 is indicated by a longitudinal axis 105, which extends from the inflow end 109 to the outflow end 108 (FIGS. 2A and 2B). Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame 102, as further described below. One or more of posts 104 can be configured as support posts 107.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In some examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As one example, the prosthetic valve 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (that is, each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in some examples.

As illustrated in FIG. 2B, the struts 112 can include a first row of struts 113 at or near the inflow end 109 of the prosthetic valve 100, a second row of struts 114 at or near the outflow end 108 of the prosthetic valve 100, and third and fourth rows of struts 115, 116, respectively, positioned axially between the first and second rows of struts 113, 114. The struts 112 can form and/or define a plurality of cells (that is, openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIGS. 2A and 2B, each second cell 118 can be disposed within one of the first cells 117 (that is, the struts 115a-116b forming the second cells 118 are disposed between the struts forming the first cells 117 (that is, the struts 113a, 113b and the struts 114a, 114b), closer to an axial midline of the frame 102 than the struts 113a-114b).

As illustrated in FIGS. 2A and 2B, the struts 112 of frame 102 can comprise a curved shape. Each first cell 117 can have an axially-extending hexagonal shape including first and second apices 119 (for example, an inflow apex 119a and an outflow apex 119b). In examples where the delivery apparatus is releasably connected to the outflow apices 119b (as described below), each inflow apex 119a can be referred to as a “distal apex” and each outflow apex 119b can be referred to as a “proximal apex”. Each second cell 118 can have a diamond shape including first and second apices 120 (for example, distal apex 120a and proximal apex 120b). In some examples, the frame 102 comprises six first cells 117 extending circumferentially in a row, six second cells 118 extending circumferentially in a row within the six first cells 117, and twelve posts 104. However, in some examples, the frame 102 can comprise a greater or fewer number of first cells 117 and a correspondingly greater or fewer number of second cells 118 and posts 104.

As noted above, some of the posts 104 can be arranged in pairs of first and second posts 122, 124. The posts 122, 124 are aligned with each other along the length of the frame 102 and are axially separated from one another by a gap G (FIG. 2B) (those with actuators 126 can be referred to as actuator posts or actuator struts). Each first post 122 (that is, the lower post shown in FIGS. 2A and 2B) can extend axially from the inflow end 109 of the prosthetic valve 100 toward the second post 124, and the second post 124 (that is, the upper post shown in FIGS. 2A and 2B) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described below.

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (for example, by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In some examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122.

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In some examples, not all pairs of posts 122, 124 need be expansion and locking mechanisms (that is, actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). Specifically, when the threaded rod 126 is rotated relative to the nut 127, the first post 122, and the second post 124, the first and second posts 122, 124 can move axially relative to one another, thereby widening or narrowing the gap G (FIG. 2B) separating the posts 122, 124, and thereby radially compressing or radially expanding the prosthetic valve 100, respectively. Thus, the gap G (FIG. 2B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (for example, the delivery apparatus 200 of FIG. 3, as described below). Second, the head portion 131 can prevent the second post 124 from moving proximally relative to the threaded rod 126 and can apply a distally directed force to the second post 124, such as when radially expanding the prosthetic valve 100. Specifically, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (for example, in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (for example, clockwise) can cause corresponding axial movement of the first and second posts 122, 124 toward one another, thereby decreasing the gap G and radially expanding the frame 102, while rotation of the threaded rod 126 in an opposite second direction causes corresponding axial movement of the first and second posts 122, 124 away from one another, thereby increasing the gap G and radially compressing the frame. When the threaded rod 126 is rotated in the first direction, the head portion 131 of the rod 126 bears against an adjacent surface of the frame (for example, an outflow apex 119b), while the nut 127 and the first post 122 travel proximally along the threaded rod 126 toward the second post 124, thereby radially expanding the frame. As the frame 102 moves from a compressed configuration to an expanded configuration, the gap G between the first and second posts 122, 124 can narrow.

When the threaded rod 126 is rotated in the second direction, the threaded rod 126 and the stopper 132 move toward the outflow end 108 of the frame until the stopper 132 abuts the inflow end 170 of the second post 124 (as shown in FIGS. 2A and 2B). Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (for example, counterclockwise) causing the stopper 132 to push against (that is, provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained above, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In some examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced above, some of the posts 104 can be configured as support posts 107. As shown in FIGS. 2A and 2B, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening 146 therein.

The commissure opening 146 (which can also be referred to herein as a “commissure window 146”) can extend radially through a thickness of the support post 107 and can be configured to accept a portion of a valvular structure 150 (for example, a commissure 152) to couple the valvular structure 150 to the frame 102. For example, each commissure 152 can be mounted to a respective commissure support structure 144, such as by inserting a pair of commissure tabs of adjacent leaflets 158 through the commissure opening 146 and suturing the commissure tabs to each other and/or the commissure support structure 144. In some examples, the commissure opening 146 can be fully enclosed by the support post 107 such that a portion of the valvular structure 150 can be slid radially through the commissure opening 146, from an interior to an exterior of the frame 102, during assembly. In the illustrated example, the commissure opening 146 has a substantially rectangular shape that is shaped and sized to receive commissure tabs of two adjacent leaflets therethrough. However, in some examples, the commissure opening can have any of various shapes (for example, square, oval, square-oval, triangular, L-shaped, T-shaped, C-shaped, etc.).

The commissure openings 146 are spaced apart about the circumference of frame 102 (or angularly spaced apart about frame 102). The spacing may or may not be even. In one example, the commissure openings 146 are axially offset from the outflow end 108 of the frame 102 by an offset distance d₃ (indicated in FIG. 2A). As an example, the offset distance d₃ may be in a range from 2 mm to 6 mm. In general, the offset distance d₃ should be selected such that when the leaflets are attached to the frame 102 via the commissure openings 146, the free edge portions (for example, outflow edge portions) of the leaflets 158 will not protrude from or past the outflow end 108 of the frame 102.

The frame 102 can comprise any number of support posts 107, any number of which can be configured as commissure support structures 144. For example, the frame 102 can comprise six support posts 107, three of which are configured as commissure support structures 144. However, in some examples, the frame 102 can comprise more or less than six support posts 107 and/or more or less than three commissure support structures 144.

The inflow end portion 138 of each support post 107 can comprise an extension 154 (show as a cantilevered strut in FIGS. 2A and 2B) that extends toward the inflow end 109 of the frame 102. Each extension 154 can comprise an aperture 156 extending radially through a thickness of the extension 154. In some examples, the extension 154 can extend such that an inflow edge of the extension 154 aligns with or substantially aligns with the inflow end 109 of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts and/or the leaflets and/or the connecting skirt 125 can be coupled. For example, sutures used to connect the inner and/or outer skirts and/or the leaflets and/or the connecting skirt 125 can be wrapped around the extensions 154 and/or can extend through apertures 156.

As an example, each extension 154 can have an aperture 156 (FIG. 2A) or other features to receive a suture or other attachment material for connecting an adjacent inflow edge portion 160 of a leaflet 158 (FIG. 1A), the outer skirt 103 (in FIG. 1B), the connecting skirt 125, and/or an inner skirt. In some examples, the inflow edge portion 160 of each leaflet 158 can be connected to a corresponding extension via a suture 135 (FIG. 1A).

In some examples, the outer skirt 103 can be mounted around the outer surface of frame 102 as shown in FIG. 1B and the inflow edge of the outer skirt 103 (lower edge in FIG. 1B) can be attached to the connecting skirt 125 and/or the inflow edge portions 160 of the leaflets 158 that have already been secured to frame 102 as well as to the extensions 154 of the frame by sutures 129. The outflow edge of the outer skirt 103 (the upper edge in FIG. 1B) can be attached to selected struts with stitches 137. In implementations where the prosthetic valve includes an inner skirt, the inflow edge of the inner skirt can be secured to the inflow edge portions 160 before securing the cusp edge portions to the frame so that the inner skirt will be between the leaflets and the inner surface of the frame. After the inner skirt and leaflets are secured in place, then the outer skirt can be mounted around the frame as described above.

The frame 102 can be a unitary and/or fastener-free frame that can be constructed from a single piece of material (for example, Nitinol, stainless steel, or a cobalt-chromium alloy), such as in the form of a tube. The plurality of cells can be formed by removing portions (for example, via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material, such as stainless steel or a cobalt-chromium alloy. When the frame is formed from a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuation assemblies of the delivery apparatus (as further described below), which rotate the rods 126 to produce expansion of the frame 102. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In some examples, the frame 102 can be formed from a self-expandable material (for example, Nitinol). When the frame 102 is formed from a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 102 (and therefore the prosthetic valve 100) can be further radially expanded from the partially expanded state to a further radially expanded state via actuation of actuation assemblies of the delivery apparatus (as further described below), which rotate the rods 126 to produce expansion of the frame.

As introduced above, the threaded rods 126 can removably couple the prosthetic valve 100 to actuator assemblies of a delivery apparatus. Referring to FIG. 3, it illustrates an example of a delivery apparatus 200 for delivering the prosthetic valve 100 to a desired implantation location. The prosthetic valve 100 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 100 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (for example, actuator or threaded rod 126) on the prosthetic valve. For example, three actuator assemblies 208 can be provided for a prosthetic valve having three actuators. In some examples, a greater or fewer number of actuator assemblies 208 can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery. It should be noted that the distal end portion of the shaft can also be used to house various other implantable devices (for example, stents, grafts, etc.).

The actuator assemblies 208 can be releasably coupled to the prosthetic valve 100. For example, in the illustrated example, each actuator assembly 208 can be coupled to a respective actuator (for example, threaded rod 126) of the prosthetic valve 100. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand and/or deploy the prosthetic valve 100. For example, in the illustrated example the handle 204 comprises first, second, and third knobs 211, 212, and 214, respectively.

The first knob 211 can be a rotatable knob configured to produce axial movement of the first shaft 206 relative to the prosthetic valve 100 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (for example, clockwise) can retract the sheath 216 proximally relative to the prosthetic valve 100 and rotation of the first knob 211 in a second direction (for example, counter-clockwise) can advance the sheath 216 distally. In some examples, the first knob 211 can be actuated by sliding or moving the first knob 211 axially, such as pulling and/or pushing the knob. In some examples, actuation of the first knob 211 (rotation or sliding movement of the first knob 211) can produce axial movement of the actuator assemblies 208 (and therefore the prosthetic valve 100) relative to the delivery sheath 216 to advance the prosthetic valve distally from the sheath 216.

The second knob 212 can be a rotatable knob configured to produce radial expansion and/or compression of the prosthetic valve 100. For example, rotation of the second knob 212 can rotate the threaded rods of the prosthetic valve 100 via the actuator assemblies 208. Rotation of the second knob 212 in a first direction (for example, clockwise) can radially expand the prosthetic valve 100 and rotation of the second knob 212 in a second direction (for example, counter-clockwise) can radially collapse the prosthetic valve 100. In some examples, the second knob 212 can be actuated by sliding or moving the second knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (for example, threaded rod). Once the actuator assemblies 208 are uncoupled from the prosthetic valve 100, the delivery apparatus 200 can be removed from the patient, leaving just the prosthetic valve 100 in the patient.

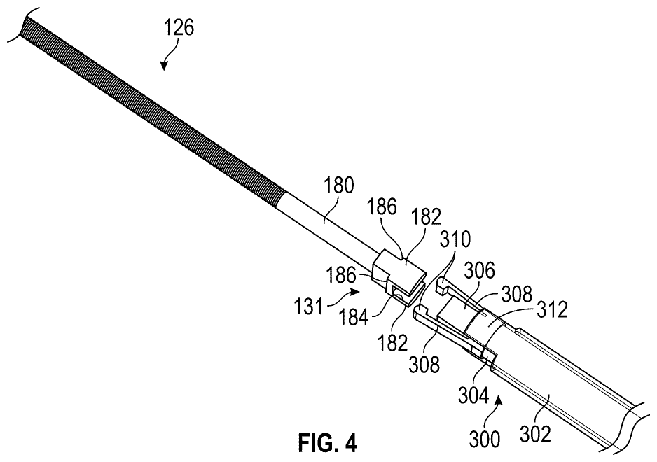

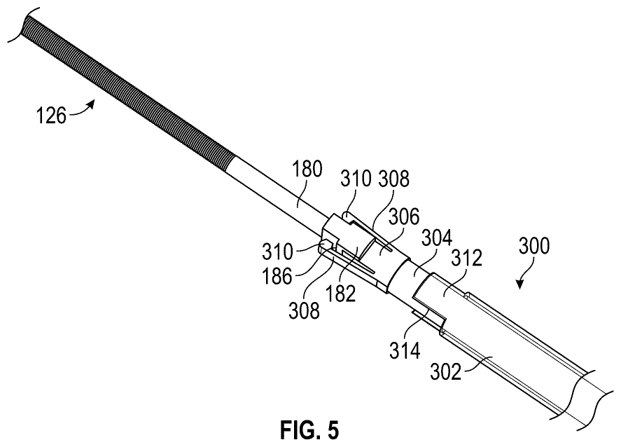

Referring to FIGS. 4-5, they illustrate how each of the threaded rods 126 of the prosthetic device 100 can be removably coupled to an actuator assembly 300 (for example, actuator assemblies 208) of a delivery apparatus (for example, delivery apparatus 200), according to one example. Specifically, FIG. 5 illustrates how one of the threaded rods 126 can be coupled to an actuator assembly 300, while FIG. 4 illustrates how the threaded rod 126 can be detached from the actuator assembly 300.

As introduced above, an actuator assembly 300 can be coupled to the head portion 131 of each threaded rod 126. The head portion 131 can be included at a proximal end portion 180 of the threaded rod 126 and can extend proximally past a proximal end of the second post 124 (FIG. 2A). The head portion 131 can comprise first and second protrusions 182 defining a channel or slot 184 between them, and one or more shoulders 186. As discussed above, the head portion 131 can have a width greater than a diameter of the inner bore of the second post 124 such that the head portion 131 is prevented from moving into the inner bore of the second post 124 and such that the head portion 131 abuts the outflow end 108 of the frame 102. In particular, the head portion 131 can abut an outflow apex 119b of the frame 102. The head portion 131 can be used to apply a distally-directed force to the second post 124, for example, during radial expansion of the frame 102.

Each actuator assembly 300 can comprise a first actuation member configured as a support tube or outer sleeve 302 and a second actuation member configured as a driver 304. The driver 304 can extend through the outer sleeve 302. The outer sleeve 302 is shown transparently in FIGS. 4-5 for purposes of illustration. The distal end portions of the outer sleeve 302 and driver 304 can be configured to engage or abut the proximal end of the threaded rod 126 (for example, the head portion 131) and/or the frame 102 (for example, the apex 119b). The proximal portions of the outer sleeve 302 and driver 304 can be operatively coupled to the handle of a delivery apparatus (for example, handle 204). The delivery apparatus in this example can include the same features described previously for delivery apparatus 200. In particular examples, the proximal end portions of each driver 304 can be operatively connected to the knob 212 such that rotation of the knob 212 (clockwise or counterclockwise) causes corresponding rotation of the drivers 304. The proximal end portions of each outer sleeve 302 can be operatively connected to the knob 214 such that rotation of the knob 214 (clockwise or counterclockwise) causes corresponding axial movement of the sleeves 302 (proximally or distally) relative to the drivers 304. In some examples, the handle can include electric motors for actuating these components.

The distal end portion of the driver 304 can comprise a central protrusion 306 configured to extend into the slot 184 of the threaded rod 126, and one or more flexible elongated elements or arms 308 including protrusions or teeth 310 configured to be releasably coupled to the shoulders 186 of the threaded rod 126. The protrusions 310 can extend radially inwardly toward a longitudinal axis of the second actuation member 304. As shown in FIGS. 4-5, the elongated elements 308 can be configured to be biased radially outward to an expanded state, for example, by shape setting the elements 308.

As shown in FIG. 5, to couple the actuator assembly 300 to the threaded rod 126, the driver 304 can be positioned such that the central protrusion 306 is disposed within the slot 184 (FIG. 4) and such that the protrusions 310 of the elongated elements 308 are positioned distally to the shoulders 186. As the outer sleeve 302 is advanced (for example, distally) over the driver 304, the sleeve 302 compresses the elongated elements 308 they abut and/or snap over the shoulders 186, thereby coupling the actuator assembly 300 to the threaded rod 126. Thus, the outer sleeve 302 effectively squeezes and locks the elongated elements 308 and the protrusions 310 of the driver 304 into engagement with (that is, over) the shoulders 186 of the threaded rod 126, thereby coupling the driver 304 to the threaded rod 126.

Because the central protrusion 306 of the driver 304 extends into the slot 184 of the threaded rod 126 when the driver 304 and the threaded rod 126 are coupled, the driver 304 and the threaded rod 126 can be rotational locked such that they co-rotate. So coupled, the driver 304 can be rotated (for example, using knob 212 the handle of the delivery apparatus 200) to cause corresponding rotation of the threaded rod 126 to radially expand or radially compress the prosthetic device. The central protrusion 306 can be configured (for example, sized and shaped) such that it is advantageously spaced apart from the inner walls of the outer sleeve 302, such that the central protrusion 306 does not frictionally contact the outer sleeve 302 during rotation. Though in the illustrated example the central protrusion 306 has a substantially rectangular shape in cross-section, in some examples, the protrusion 306 can have any of various shapes, for example, square, triangular, oval, etc. The slot 184 can be correspondingly shaped to receive the protrusion 306.

The outer sleeve 302 can be advanced distally relative to the driver 304 past the elongated elements 308, until the outer sleeve 302 engages the frame 102 (for example, a second post 124 of the frame 102). The distal end portion of the outer sleeve 302 also can comprise first and second support extensions 312 defining gaps or notches 314 between the extensions 312. The support extensions 312 can be oriented such that, when the actuator assembly 300 is coupled to a respective threaded rod 126, the support extensions 312 extend partially over an adjacent end portion (for example, the upper end portion) of one of the second posts 124 on opposite sides of the post 124. The engagement of the support extensions 312 with the frame 102 in this manner can counter-act rotational forces applied to the frame 102 by the rods 126 during expansion of the frame 102. In the absence of a counter-force acting against these rotational forces, the frame can tend to “jerk” or rock in the direction of rotation of the rods when they are actuated to expand the frame. The illustrated configuration is advantageous in that outer sleeves, when engaging the proximal posts 124 of the frame 102, can prevent or mitigate such jerking or rocking motion of the frame 102 when the frame 102 is radially expanded.

To decouple the actuator assembly 300 from the prosthetic device 100, the sleeve 302 can be withdrawn proximally relative to the driver 304 until the sleeve 302 no longer covers the elongated elements 308 of the driver 304. As described above, the sleeve 302 can be used to hold the elongated elements 308 against the shoulders 186 of the threaded rod 126 since the elongated elements 308 can be naturally biased to a radial outward position where the elongated elements 308 do not engage the shoulders 186 of the threaded rod 126. Thus, when the sleeve 302 is withdrawn such that it no longer covers/constrains the elongated elements 308, the elongated elements 308 can naturally and/or passively deflect away from, and thereby release from, the shoulders 186 of the threaded rod 126, thereby decoupling the driver 304 from the threaded rod 126.

The sleeve 302 can be advanced (moved distally) and/or retracted (moved proximally) relative to the driver 304 via a control mechanism (for example, knob 214) on the handle 204 of the delivery apparatus 200, by an electric motor, and/or by another suitable actuation mechanism. For example, the physician can turn the knob 214 in a first direction to apply a distally directed force to the sleeve 302 and can turn the knob 214 in an opposite second direction to apply a proximally directed force to the sleeve 302. Thus, when the sleeve 302 does not abut the prosthetic device and the physician rotates the knob 214 in the first direction, the sleeve 302 can move distally relative to the driver 304, thereby advancing the sleeve 302 over the driver 304. When the sleeve 302 does abut the prosthetic device, the physician can rotate the knob 214 in the first direction to push the entire prosthetic device distally via the sleeve 302. Further, when the physician rotates the knob 214 in the second direction the sleeve 302 can move proximally relative to the driver 304, thereby withdrawing/retracting the sleeve 302 from the driver 304.

FIG. 6 illustrates an example of a delivery apparatus 400. The delivery apparatus 400 can, for example, provide for manipulation of a radius of curvature of a shaft of the delivery apparatus 400 independent of an axial displacement of the shaft relative to other components of the delivery apparatus. For example, the shaft of the delivery apparatus 400 can be retracted relative to a prosthetic implant coupled to the delivery apparatus 400 via a shaft displacement mechanism, without adjusting the radius of curvature of the shaft. Similarly, the delivery apparatus 400 can enable the curvature of the shaft to be adjusted via a shaft adjustment mechanism, without changing the axial position of the shaft relative to the prosthetic implant.

Similar to delivery apparatus 200, a prosthetic valve (for example, mechanically-expandable prosthetic valves such as prosthetic valve 100 described herein, self-expandable prosthetic valves, balloon-expandable prosthetic valves, etc.) can be releasably coupled to the delivery apparatus 400. It should be understood that the delivery apparatus 400 and other delivery apparatuses disclosed herein can be used to implant prosthetic devices other than prosthetic valves, such as stents or grafts.

The delivery apparatus 400 in the illustrated example generally includes a handle 404, a first elongated shaft 406 extending distally from the handle 404 and at least one expansion mechanism 408 extending distally through the first shaft 406 (FIG. 7). Although not shown, a prosthetic device, such as a prosthetic heart valve, can be coupled to the expansion mechanism 408. The expansion mechanism 408 can include be one of the expansion mechanisms described herein (for example, one or more of actuator assemblies described herein, such as, a balloon for a balloon-expandable prosthetic device, an inner shaft having a self-expandable prosthetic device disposed on an outer surface thereof, etc.) or other types of expansion mechanism suitable for an expandable prosthetic device.

In some examples, a distal end portion of the shaft 406 can be sized to house the prosthetic device in its radially compressed, delivery state (for example, as coupled to the expansion mechanism 408) during delivery of the prosthetic valve through the patient's vasculature. In this manner, the distal end portion of the shaft 406 functions as a delivery sheath or capsule for the prosthetic valve during delivery. Further details regarding delivery capsules and retraction of delivery capsules can be found, for example, in U.S. Provisional Application No. 63/322,974, filed Mar. 23, 2022, which is incorporated by reference herein.

The handle 404 of the delivery apparatus 400 can include one or more control mechanisms (for example, knobs or other actuating mechanisms) for controlling different components of the delivery apparatus 400 in order to expand and/or deploy the prosthetic valve. For example, in the illustrated example the handle 404 comprises first, second, and third knobs 411, 412, and 414, respectively.

The first knob 411 (also referred to herein as a “flex knob”) can be a rotatable knob configured to aid in advancing the delivery shaft 406 to and/or positioning the delivery shaft 406 at a location at or adjacent a desired implantation location with the patient's body. For example, the first knob 411 is configured to be adjusted by the user to flex, bend, twist, turn, and/or otherwise articulate the distal end portion of the delivery shaft 406 to aid in advancing and/or positioning the delivery shaft 406 for deployment of the prosthetic valve at the implantation site. For example, rotation of the first knob 411 in a first direction (for example, clockwise) relative to the handle 404 can increase the curvature of the shaft 406 and rotation of the first knob 411 in a second direction (for example, counterclockwise) relative to the handle 404 can decrease the curvature of the shaft 406. In some examples, the first knob 411 can be actuated by sliding or moving the first knob 411 axially, such as pulling and/or pushing the knob.

The second knob 412 (also referred to herein as a “shaft displacement knob”) can be a rotatable knob configured to produce axial movement of the first shaft 406 relative to the prosthetic valve in the distal and/or proximal directions in order to deploy the prosthetic valve from the delivery shaft 406 once the prosthetic valve has been advanced to the location at or adjacent the desired implantation location. For example, rotation of the second knob 412 in a first direction (for example, clockwise) relative to the handle 404 can retract the shaft 406 proximally relative to the prosthetic valve and rotation of the second knob 412 in a second direction (for example, counter-clockwise) relative to the handle 404 can advance the shaft 406 distally. In some examples, the second knob 412 can be actuated by sliding or moving the second knob 412 axially, such as pulling and/or pushing the knob. In some examples, actuation of the second knob 412 (rotation or sliding movement of the second knob 412) can produce axial movement of the expansion mechanisms 408 (and therefore the prosthetic valve) relative to the shaft 406 to advance the prosthetic valve distally from the shaft 406.

The third knob 414 (also referred to herein as an “actuation knob”) can be a rotatable knob configured to produce radial expansion and/or compression of the prosthetic valve. For example, in connection with mechanically-expandable prosthetic devices, rotation of the third knob 414 can rotate actuators of the prosthetic valve via the expansion mechanisms 408. Rotation of the third knob 414 in a first direction (for example, clockwise) relative to the handle 404 can radially expand the prosthetic valve and rotation of the third knob 414 in a second direction (for example, counter-clockwise) relative to the handle 404 can radially collapse the prosthetic valve. In some examples, in connection with balloon-expandable prosthetic devices, rotation of the third knob 414 can result in inflation of a balloon expansion mechanism 408. In some examples, the third knob 414 can be omitted, for example, in connection with self-expandable prosthetic devices. In some examples, the third knob 414 can be actuated by sliding or moving the third knob 414 axially, such as pulling and/or pushing the knob.

The handle 404 of the delivery apparatus 400 can include one or more indicator mechanisms, such as indicator 416. The indicator 416 (also referred to herein as a “flex indicator”) can be operatively coupled to the first knob 411 and can be configured to indicate an amount of flex or curvature of the shaft 406 as the first knob 411 is rotated, as described in more detail below. As shown, the indicator 416 can include indicia, such as alphanumeric characters, laterally aligned hashmarks, graphics, etc. to visually indicate the amount of curvature of the shaft 406.

As shown in FIG. 6, the indicator 416 is positioned at a distal end 418 of the handle 404. In some examples, as depicted, the first knob 411 can be positioned proximal to the indicator 416. The second knob 412 can be positioned proximal to the first knob 411. The third knob 414 can be positioned at a proximal end 420 of the handle 404 and can be proximal to the first and second knobs 411, 412. In some examples, the indicator 416 and the knobs 411, 412, and 414 may be arranged in a different order, for example, with the third knob 414 positioned at the distal end 418 of the handle 404.

The handle 404 can also include an outer housing 422. As shown in FIG. 7, within the housing 422 and/or within one or more of the knobs 411, 412, etc., the handle 404 can include the expansion mechanisms 408, an adjustment mechanism 424 for adjusting the flex or curvature of the delivery shaft 406, and a displacement mechanism 426 for axially displacing the delivery shaft 406 relative to the expansion mechanisms 408. In some instances, the housing 422 can be integrally formed as a single, unitary component. In other instances, the housing 422 can comprise one or more segments that are formed as separate components that are coupled together (for example, via fasteners, adhesive, mating features, and/or other means for coupling).

In some examples, as depicted, the adjustment mechanism 424 can be coupled to the displacement mechanism 426. Specifically, the delivery apparatus 400 can include a connector shaft 428 to couple the adjustment mechanism 424 and the displacement mechanism 426.

The adjustment mechanism 424 can be operatively coupled to the first knob 411. In some examples, the distal end portion of the delivery shaft 406 can be configured to be steerable via the adjustment mechanism 424 based on rotation of the first knob 411 relative to the housing 422. For example, by rotating the knob 411, a curvature of the distal end portion of the delivery shaft 406 can be adjusted so that the distal end portion of the delivery shaft 406 can be oriented in a desired angle. Specifically, to implant a prosthetic device (for example, prosthetic valve 100, prosthetic valve 100, etc.), the distal end portion of the delivery shaft 406 can be steered so that the prosthetic valve can be positioned at a target implantation location.

In addition to the knob 411, the adjustment mechanism 424 (also referred to herein as a “flex assembly”) can include a pull wire 430, as shown in FIG. 7. The adjustment mechanism 424 can be configured to steer the distal end portion of the delivery shaft 406 via the knob 411 and the pull wire 430 by increasing or decreasing the tension of the pull wire 430. Specifically, a distal end of the pull wire 430 can be connected to the distal end portion of the delivery shaft 406. When the tension of the pull wire 430 changes, the curvature of the distal end portion of the delivery shaft 406 changes in response to the tension of the pull wire 430.

The adjustment mechanism 424 can also include a flex nut 432 and a flex lead member 434 to axially displace the pull wire 430 relative to the handle 404. For example, the pull wire 430 can extend proximally into the handle 404 and a proximal end of the pull wire 430 can be connected to the flex nut 432 (see FIG. 9). As described in more detail below, the flex nut 432 can be configured to translate axially relative to the flex lead member 434.

In some examples, the delivery apparatus 400 can also include one or more gear assemblies 472 to couple the knob 411 to the other components of the adjustment mechanism 424 disposed within the handle 404, as described in more detail below. For example, each gear assembly 472 can include one or more first (or proximal) gears 436, one or more rods 438, and one or more second (or distal) gears 440.

As depicted, the gear assemblies 472 enable the flex nut 432 and the flex lead member 434 of the adjustment mechanism 424 to be disposed proximal (or move proximally) to at least some of the components of the displacement mechanism 426 within the handle 404 (such as a carriage 464 of the displacement mechanism 426). The gear assemblies 472 can enable the adjustment mechanism 424 to be disposed generally proximal to the displacement mechanism 426, even though the knob 411 corresponding to the adjustment mechanism 424 is distal to the knob 412 corresponding to the displacement mechanism 426. For example, the rods 438 can generally extend at least a portion of the length of the handle 404 with the rods 438 extending proximally from the gears 440, through the carriage 464 of the displacement mechanism 426, and at least to the gears 436 which can be coupled to the flex lead member 434 of the adjustment mechanism 424. In some examples, the rods 438 do not need to extend through the carriage 464 of the displacement mechanism 426.

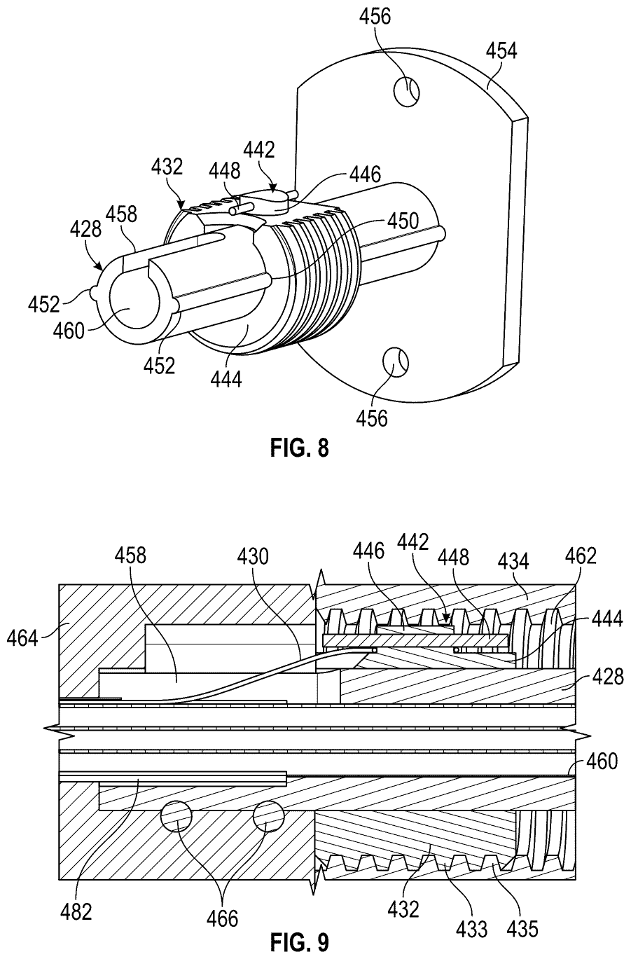

As shown in FIGS. 8-9, the flex nut 432 includes an attachment member 442 (also referred to as a “wire wrap”) for coupling the pull wire 430 to the flex nut 432. The attachment member 442 can be configured to secure a proximal end of the pull wire 430 thereto (for example, by wrapping an end of the pull wire 430 around the attachment member 442, etc.). As shown, the attachment member 442 extends from a main body 444 of the flex nut 432 in a radial direction. In some examples, as depicted, the attachment member 442 can include a radial projection 446 and a pin 448 extending axially from the projection 446. In this way, the pin 448 can be radially offset from the main body 444. The pull wire 430 can be wrapped around the projection 446 and the pin 448 to couple the pull wire 430 to the attachment member 442. In some examples, wrapping the pull wire 430 around the pin 448 can result in plastic deformation of the pull wire 430. In this way, the plastic deformation of the pull wire 430 can help to prevent the pull wire 430 from disconnecting or unwrapping from the attachment member 442.

In some examples, the pull wire can be coupled to the flex nut in various other ways. For example, fasteners, adhesive, and/or other means of coupling can be used to couple the pull wire to the flex nut.

The flex nut 432 can be disposed around the connector shaft 428, such that the connector shaft 428 extends through an opening of the flex nut 432. The flex nut 432 can be configured to translate axially relative to the connector shaft 428 to adjust the tension of the pull wire 430. In some examples, as described in more detail below, rotation of the flex lead member 434 can result in the axial translation of the flex nut 432 relative to the connector shaft 428. Each flex nut 432 can include one or more notches 450 which can be configured to prevent rotational movement of the flex nut 432 relative to the connector shaft 428 during axial translation of the flex nut 432 along the connector shaft 428. In particular, the notches 450 of the flex nut 432 can engage with guides 452 of the connector shaft 428 as the flex nut 432 is axially moved by the flex lead member 434. In this manner, the guides 452 can prevent rotational movement of the flex nut 432, while allowing the flex nut 432 to move in an axial direction relative to the connector shaft 428 and flex lead member 434.

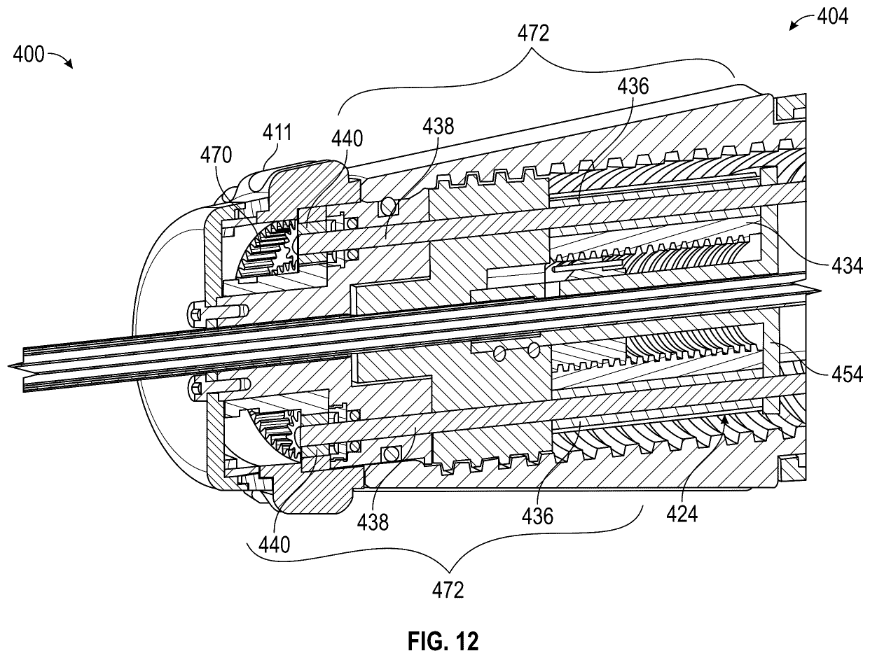

As shown in FIG. 8, the guides 452 can be projections from the outer surface of the connector shaft 428 that extend along a length of the connector shaft 428. For example, the guides 452 can extend from a distal end of the connector shaft 428 to a flange 454 at the proximal end of the connector shaft 428. The flange 454 can include openings 456 for the rods 438 to pass therethrough (for example, as shown in FIG. 12). While two notches 450 and two corresponding guides 452 are shown in the illustrated example, greater or fewer notches 450 and guides 452 may be included in some examples.

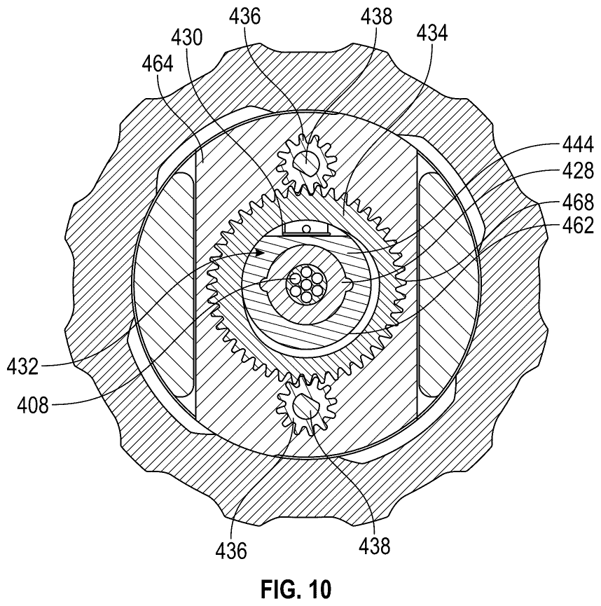

The connector shaft 428 can include an axially extending slot 458 at the distal end to allow the pull wire 430 to pass from a location that is radially inward of the connector shaft 428 (for example, the distal end of the delivery shaft 406) to a location that is radially outward of the connector shaft 428 (for example, attachment member 442). In some examples, as depicted, the connector shaft 428 includes a central lumen 460. As shown in FIG. 10, for example, the expansion mechanisms 408 are disposed within and extend through the lumen 460.

In some examples, as shown in FIG. 9, the main body 444 of the flex nut 432 includes external threads 433 that can be mated with internal threads 435 of the flex lead member 434. Specifically, the flex lead member 434 can include an inner surface 462 defining a lumen and the internal threads 435, and the external threads 433 of the flex nut 432 can be threadedly engaged with the threads 435 of the inner surface 462. As described in more detail below, the flex nut 432 is permitted to translate axially within the flex lead member 434 along the threads 435 of the inner surface 462 and along the connector shaft 428, based on rotation of the first knob 411 relative to the handle 404.

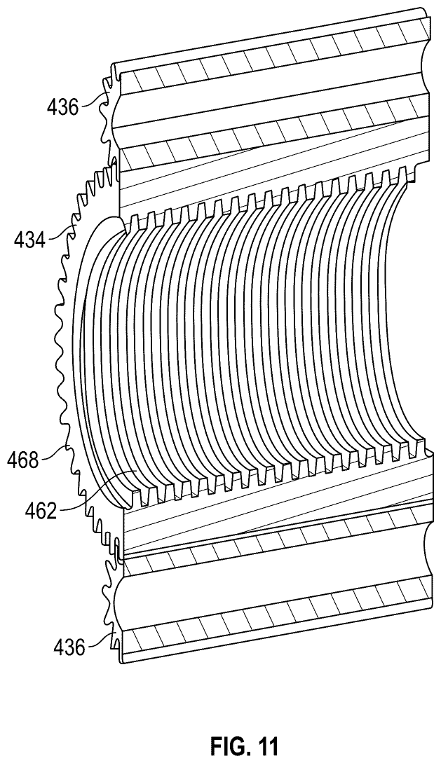

As introduced above, the connector shaft 428 can be coupled to the adjustment mechanism 424 and the displacement mechanism 426. Specifically, a distal end of the connector shaft 428 can be coupled to a carriage 464 of the displacement mechanism 426. In some examples, as depicted, pins 466 are used to couple the connector shaft 428 to the carriage 464. In some examples, the connector shaft 428 can be coupled to the carriage 464 in other manners, such as with an adhesive, a friction fit, or other coupling mechanisms. At the proximal end of the connector shaft 428, the flange 454 can be coupled to the rods 438 and the flange 454 can be adjacent to proximal ends of the flex lead member 434 and the proximal gears 436 (for example, as shown in FIG. 12). In some examples, the flex lead member 434 can be disposed around the connector shaft 428 and the rods 438 can extend through the openings 456 of the flange 454 (for example, as shown in FIG. 12). The rods 438 can be rotatable relative to the connector shaft 428 within the opening 456. The proximal gears 436 can be disposed on the rods 438 and can be coupled (for example, rotatably coupled) to an outer surface of the flex lead member 434 (for example, as shown in FIG. 11). In some examples, the proximal gears 436 are fixedly coupled to the rods 438.

The flex lead member 434 (also referred to herein as a “barrel” or an “adjustment barrel”) can include inner surface 462 and an outer surface 468, as shown in FIG. 10. In some examples, as depicted, the inner surface 462 is threaded and the outer surface 468 includes outer teeth, as shown in FIG. 11. As described above, the main body 444 of the flex nut 432 includes external threads 433 which engage with the internal threads 435 of the flex lead member 434, as shown in FIG. 9. The teeth of the outer surface 468 are meshed with teeth of the proximal gears 436. As such, when the proximal gears 436 are rotated by rotation of the rods 438, the flex lead member 434 is also rotated. Due to the threaded connection between the flex lead member 434 and the flex nut 432 (that is, connection between the external threads 433 of the flex nut 432 and the internal threads 435 of the flex lead member 434), the flex nut 432 (and therefore, the pull wire 430) is displaced axially (along the connector shaft 428 and within the flex lead member 434) to change the tension of the pull wire 430 and adjust the curvature or flex of the distal end portion of the delivery shaft 406.

In some examples, as depicted in FIGS. 7 and 11, the proximal gears 436 extend axially along the rods 438 and span at least a portion of a length of the flex lead member 434. The length of the proximal gears 436 can be equal to an axial length between the carriage 464 to the flange 454. In some examples, the proximal gears 436 do not extend the entire axial length of the flex lead member 434. In some examples, one or more gears 436 that are smaller in the axial direction may be mounted on the rods 438 and coupled to the outer surface 468 of the flex lead member 434 (for example, between the carriage 464 and the flange 454 in the axial direction). Additionally, one or more optional spacers may be mounted on the rods 438 between the carriage 464, the gear(s), and/or the flange 454 in the axial direction. In some instances, the total axial length of the gear(s) and the spacer(s) can equal the axial length of the flex lead member 434.

The proximal gears 436 and the distal gears 440 can be coupled to the rods 438, such that the gears 436, 440 are not permitted to rotate relative to the rods 438. For example, the rods 438 can be shaped (for example, D-shaped) such that the rods 438 mate with a corresponding opening of the gears 436, 440.

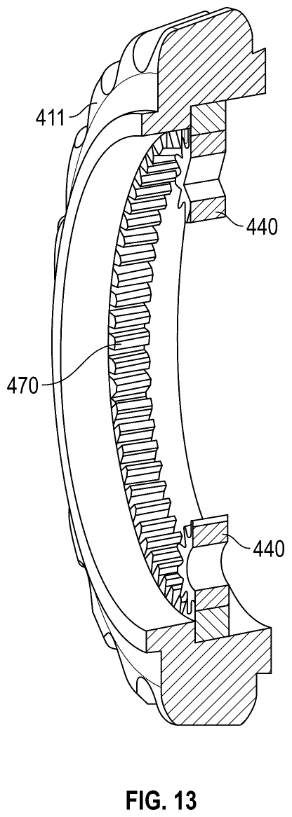

As shown in FIGS. 12 and 13, the knob 411 is coupled to the rods 438 via the distal gears 440. Specifically, the knob 411 includes inner teeth 470 that are meshed with (for example, engages with) teeth of the distal gears 440. In some examples, as depicted in FIG. 12, the adjustment mechanism 424 includes two groupings of one proximal gear 436, one rod 438, and one distal gear 440. For example, each grouping 472 (also referred to as “gear assembly 472”) is disposed in a circumferentially spaced apart manner, such that the gear assemblies 472 are equally spaced around the handle 404. While two gear assemblies 472 are included in the illustrated example, a different number of gear assemblies 472 (for example, one, three, four, etc.) can be included in other examples. In some examples, each gear assembly 472 can include a different number of gears and/or rods (for example, two proximal gears 436, one rod 438, one distal gear 440, etc.). In some examples, each gear assembly 472 can include a different configuration of gears or rods that operatively couple the knob 411 to the flex lead member 434.

As introduced above, the delivery apparatus 400 can also include a displacement mechanism 426 configured to axially displace delivery shaft 406 relative to the expansion mechanisms 408 (for example, to retract the delivery shaft 406 relative to a prosthetic heart valve coupled to the expansion mechanisms 408). Referring again to FIG. 7, the displacement mechanism 426 can include the second knob 412 and the carriage 464 (also referred to herein as a “displacement nut 464” or “displacement member 464”). As described in more detail below, rotation of the knob 412 relative to the handle 404 can drive axial displacement of the carriage 464 (and the shaft 406 coupled thereto) relative to the handle 404.

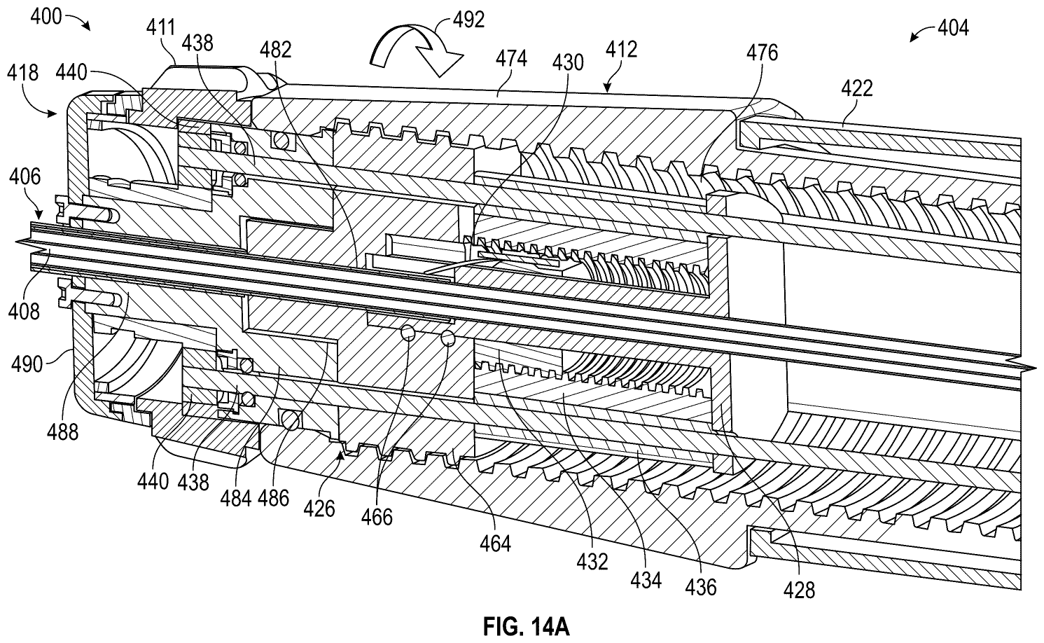

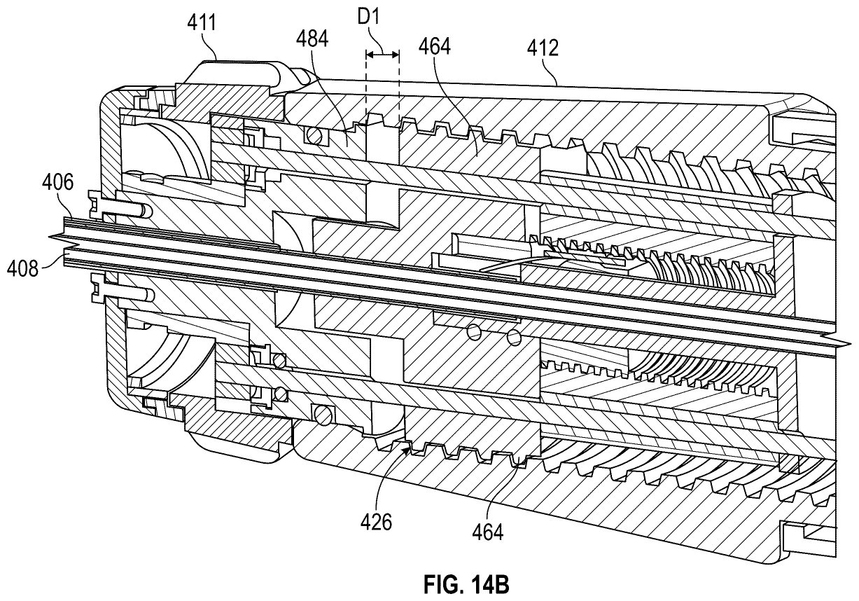

The knob 412 can include an outer grip portion 474 and a barrel portion 476 (for example, as shown in FIGS. 7 and 14A). The grip portion 474 is configured to be engaged by a user to rotate the knob 412 relative to the handle 404. Rotation of knob 412 (and therefore the barrel portion 476) is configured to axially displace one or more components of the shaft displacement mechanism 426 (for example, the displacement nut 464) relative to the barrel portion 476. In some examples, as depicted, the barrel portion 476 can be disposed radially within the grip portion 474 and can extend proximally from the grip portion 474 within the outer housing 422. As shown in FIG. 7, the displacement nut 464, the connector shaft 428, and one or more components of the adjustment mechanism 424 can be disposed within the barrel portion 476 of the knob 412. In some examples, as depicted, the knob 412 can be integrally formed as a single, unitary component. In some examples, the knob 412 can comprise one or more segments that are formed as separate components that are coupled together (for example, via fasteners, adhesive, mating features, and/or other means for coupling). For example, each of the grip portion 474 and the barrel portion 476 may be formed as separate components that are coupled together.

The barrel portion 476 can include a threaded inner surface 478 and the displacement nut 464 can include a corresponding threaded outer surface 480. The displacement nut 464 can be disposed within the barrel portion 476, with the threaded outer surface 480 coupled to the inner surface 478. In this way, rotation of the knob 412 can cause the displacement nut 464 to translate axially within the barrel portion 476 via the threaded connection of the surfaces 478, 480. In some examples, as shown, the displacement nut 464 can include openings through which the rods 438 extend. When the displacement nut 464 is axially displaced, for example, as a result of rotation of the knob 412, the nut 464 translates axially along the rods 438. In addition to the displacement nut 464 being configured to translate axially relative to the rods 438, the rods 438 can be rotatable within the openings of the displacement nut 464, for example, as a result of rotation of the first knob 411.

The displacement nut 464 can be coupled to a proximal end portion 482 of the delivery shaft 406. In this way, displacement of the nut 464 results in displacement of the delivery shaft 406.

In some examples, as depicted in FIG. 9, the proximal end portion 482 of the delivery shaft 406 can also be coupled to the connector shaft 428. For example, the proximal end portion 482 of the delivery shaft 406 can be positioned within the lumen 460 of the connector shaft 428 and coupled thereto (for example, with an adhesive, etc.). In such instances, the connector shaft 428 can be coupled to the displacement nut 464, for example, via pins 466. In this way, the relative axial positioning of the delivery shaft 406, the displacement nut 464, and the connector shaft 428 can be fixed such that axial displacement of one of these components results in axial displacement of the others.

For example, rotation of the knob 412 can cause the displacement nut 464 to translate axially within the barrel portion 476 via the threaded connection of the surfaces 478, 480, and this translation can result in axial translation of the delivery shaft 406 and the connector shaft 428. Specifically, the displacement nut 464 can be configured to translate axially relative to the expansion mechanisms 408, such that the delivery shaft 406 can be retracted relative to the expansion mechanisms 408.

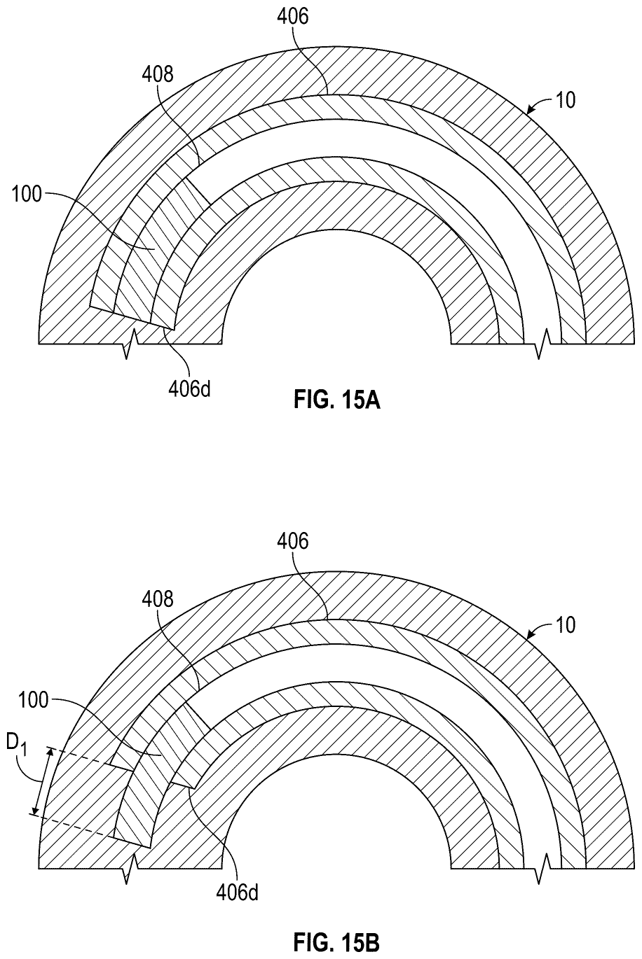

FIGS. 14A-15B illustrate operation of the displacement mechanism 426 in greater detail. Specifically, FIGS. 14A-14B illustrate operation of the components of the displacement mechanism 426 disposed within the handle 404, and FIGS. 15A-15B schematically illustrate displacement of the delivery shaft 406 relative to the prosthetic valve 100 that results from the operation depicted in FIGS. 14A-14B.

FIG. 14A illustrates the displacement nut 464 in a first axial position within the handle 404, for example, prior to rotation of the knob 412 in a first direction relative to the handle 404. FIG. 14B illustrates the displacement nut 464 in a second axial position within the handle 404, for example, after rotation of the knob 412 in the first direction. As shown, the first axial position is distal to the second axial position. In some examples, rotation of the knob 412 in the first direction can result in proximal translation of the displacement nut 464, while rotation of the knob 412 in a second direction (for example, opposite from the first direction) relative to the handle 404 can result in distal translation of the displacement nut 464.

When the displacement nut 464 is in the first axial position (FIG. 14A), the distal end 406d of the delivery shaft 406 is in a first position relative to the prosthetic valve 100 and the expansion mechanisms 408 (FIG. 15A). When the displacement nut 464 is in the second axial position (FIG. 14B), the distal end 406d of the delivery shaft 406 is in a second position relative to the prosthetic valve 100 and the expansion mechanisms 408 (FIG. 15B). In the illustrated example, the delivery shaft 406 is partially retracted relative to the prosthetic valve 100 in the second position. As explained in more detail below, the shaft displacement mechanism 426 operates independently of the shaft adjustment mechanism 424, such that the shaft displacement mechanism 426 can be operated without adjusting the curvature of the shaft 406. For example, the curvature of the delivery shaft 406 is substantially the same in the first position (FIG. 15A) as in the second position (FIG. 15B). This can be useful, for example, when the prosthetic valve 100 is to be implanted at a target location that requires the delivery shaft 406 to be curved during implantation of the prosthetic valve 100, such as when the delivery shaft 406 is positioned within an aortic arch 10.

When in the first axial position, a distal surface of the displacement nut 464 can abut a base member 484 of the handle 404 (as shown in FIG. 14A). In some examples, as depicted, the base member 484 can include a recess 486, such that at least a portion of the displacement nut 464 can nest within the recess 486 when in the first axial position. A distal end portion 488 of the base member 484 can be coupled to a cap 490 at the distal end 418 of the handle 404. The axial position of the base member 484 and the cap 490 can be fixed relative to the handle 404 (for example, fixed relative to knobs 411, 412, to housing 422, to expansion mechanisms 408, etc.).

In some examples, as depicted, the gears 440 can be positioned adjacent to a distal surface of the base member 484 and circumferentially disposed around the distal end portion 488 of the base member 484. The rods 438, which are coupled to the gears 440, can extend through the base member 484 in some instances. Specifically, as shown in FIG. 14A, the base member 484 can include openings through which the rods 438 extend. Rods 438 are rotatable relative to the base member 484, for example, as a result of rotation of the first knob 411.

To adjust the axial positioning of the distal end 406d of the delivery shaft 406 relative to the expansion mechanisms 408 and the prosthetic valve 100, the knob 412 can be rotated relative to handle 404. For example, a user can engage the grip portion 474 to rotate the knob 412. Upon rotation of the knob 412 in a first direction 492, the threaded connection between the barrel portion 476 of the knob 412 and the displacement nut 464 can drive the displacement nut 464 in an axial direction such that the displacement nut 464 is translated (for example, proximally) along the rods 438. Due to the proximal end portion 482 of the delivery shaft 406 being fixedly coupled to the displacement nut 464, the shaft 406 is also translated axially (for example, proximally), relative to the prosthetic valve 100 and the expansion mechanisms 408. As shown in FIGS. 15A-15B, the shaft 406 translating axially enables the prosthetic valve 100 (and in some examples, the expansion mechanisms 408) to be unsheathed from the distal end 406d of the shaft 406, for example, to enable radial expansion of the prosthetic valve 100 coupled to the expansion mechanisms 408.

Further, due to the pinned connection 466 between the displacement nut 464 and the connector shaft 428, the connector shaft 428 and components of the adjustment mechanism 424 are also driven axially by the displacement nut 464. For example, rotation of the second knob 412 in the first direction 492 can result in axial displacement (for example, proximal displacement) of the connector shaft 428, the pull wire 430, the flex nut 432, the flex lead member 434, and the proximal gears 436. As such, when the knob 412 is rotated, the shaft 406 and the pull wire 430 (which is coupled to the distal end of the shaft 406) are both axially displaced by the same amount (equal to distance D1). This enables the axial position of the shaft 406 to be adjusted relative to the expansion mechanisms 408 (and the prosthetic valve 100) without changing the tension of the pull wire 430 and/or the radius of curvature of the distal end of the shaft 406. This can, for example, allow the prosthetic valve 100 to be deployed from the distal end of the shaft 406 without changing the curvature of the shaft 406, as shown in FIGS. 15A-15B. As such, the delivery apparatus 400 can, among other things, improve the process of implanting a prosthetic device and/or reduce the time of the implantation procedure.

As shown in FIG. 14B, after the knob 412 is rotated in the first direction 492, the nut 464 is displaced (for example, proximally) relative to the base member 484 by a distance D1. This distance can be equal to the amount of displacement of the distal end 406d of the shaft 406 relative to the prosthetic valve 100 (FIG. 15B). In some examples, to fully retract the shaft 406 relative to the prosthetic valve 100 coupled to the distal end portion of the expansion mechanisms 408 (“full capsule removal”), the knob 412 can be rotated in the first direction 492 by a number of revolutions, for example, one or more revolutions, etc. As discussed above, rotation of the knob 412 in a second direction relative to the handle 404 can result in distal translation of the displacement nut 464, for example, to advance the shaft 406 over the prosthetic device and/or expansion mechanisms 408.

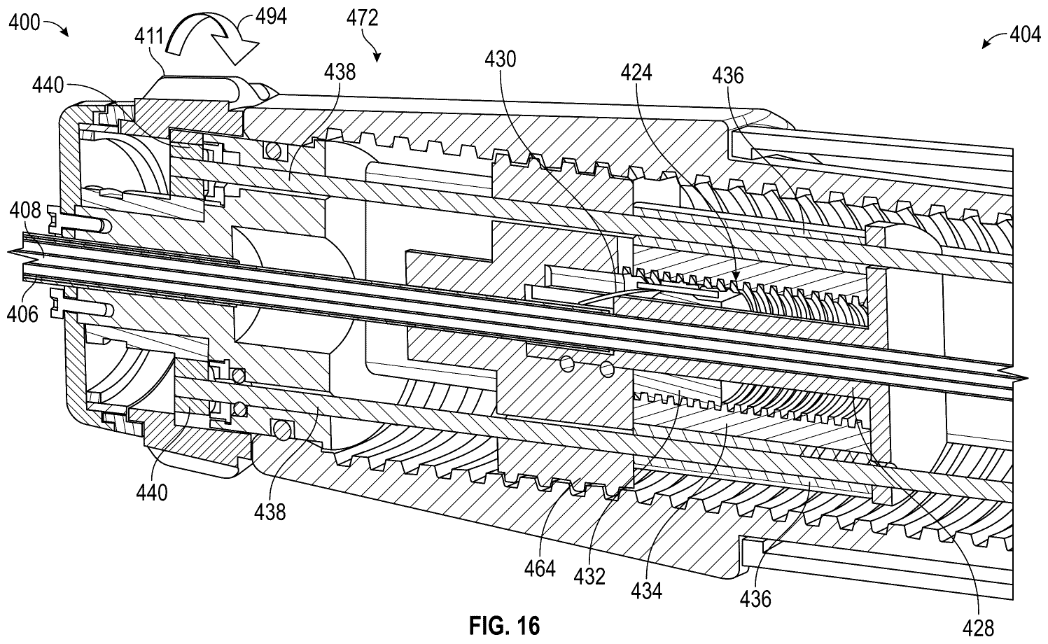

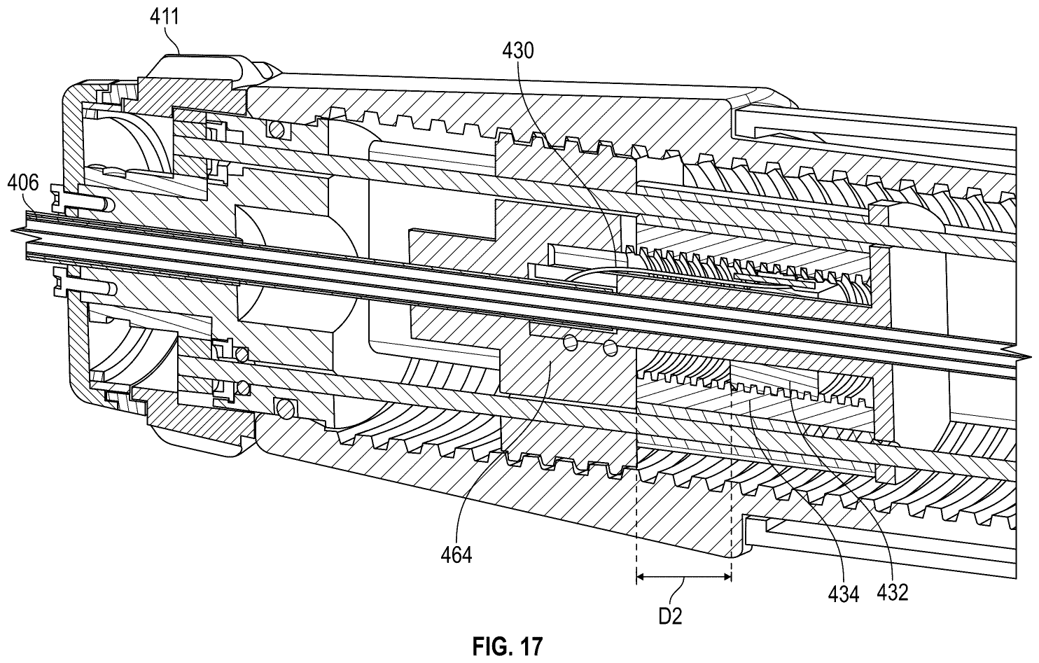

FIGS. 16-17 illustrate operation of the adjustment mechanism 424 in greater detail. Specifically, FIG. 16 illustrates the flex nut 432 in a first axial position relative to the flex lead member 434, for example, prior to rotation of the knob 411 in a first direction relative to the handle 404. FIG. 17 illustrates the flex nut 432 in a second axial position relative to the flex lead member 434, for example, after rotation of the knob 411 in the first direction. As shown, the first axial position is distal to the second axial position. In some examples, rotation of the knob 411 in the first direction relative to the handle 404 can result in proximal translation of the flex nut 432, while rotation of the knob 411 in a second direction (for example, opposite from the first direction) relative to the handle 404 can result in distal translation of the flex nut 432. In FIGS. 16-17, the displacement nut 464 is illustrated in a third axial position that is proximal to the first and second axial positions discussed with reference to FIGS. 14A and 14B. It should be appreciated that operation of the adjustment mechanism 424 is the same, regardless of the axial position of the displacement nut 464 (for example, first axial position (FIG. 14A), second axial position (FIG. 14B), etc.).

To adjust a radius of curvature of the delivery shaft 406, the knob 411 can be rotated relative to handle 404. Upon rotation of the knob 411 in a first direction 494, the gear assemblies 472 can drive rotation of the flex lead member 434 relative to the handle 404. Specifically, rotation of the knob 411 drives rotation of gears 440 which are coupled to the knob 411. The rods 438 are coupled to the gears 436, 440 such that the rods 438 and the gears 436, 440 co-rotate. In this way, as the gears 440 are rotated, the rods 438 and the gears 436 are likewise rotated. Furthermore, as discussed herein, rotation of the gears 436 causes rotation of the flex lead member 434 due to engagement between teeth of the outer surface 468 of the flex lead member 434 and teeth of the proximal gears 436, as shown in FIG. 11. Due to the threaded connection between the flex lead member 434 and the flex nut 432, rotation of the flex lead member 434 drives axial displacement of the flex nut 432 relative to the flex lead member 434 and along the connector shaft 428. Because the proximal end of the pull wire 430 is fixedly coupled to the flex nut 432, the proximal end of the pull wire 430 is also translated axially (relative to the shaft 406). This enables the tension of the pull wire 430 to be increased as the flex nut 432 and pull wire 430 are advanced proximally, for example, to increase a radius of curvature of the distal end portion of the shaft 406.

In the first axial position, a distal surface of the flex nut 432 can abut the displacement nut 464, such that the flex nut 432 is disposed at a distal end of the flex lead member 434. As shown in FIG. 17, after the knob 411 is rotated in the first direction 494, the flex nut 432 is displaced relative to the displacement nut 464 (and relative to the distal end of the flex lead member 434) by a distance D2. The tension of the pull wire 430 and the radius of curvature of the shaft 406 is dependent on this distance D2. In some examples, to fully flex the shaft 406 to a maximum radius of curvature, the knob 411 can be rotated in the first direction 494 by a number of revolutions, for example, one or more revolutions, etc. As discussed above, rotation of the knob 411 in a second direction relative to the handle 404 can result in translation of the flex nut 432 from the second axial position to the first axial position, for example, to decrease the tension of the pull wire 430 and decrease the radius of curvature of the shaft 406.

As shown in FIGS. 18-21, the knob 411 (which controls operation of the adjustment mechanism 424, as described above) can be operatively coupled to the indicator 416. Specifically, the knob 411 (and therefore the adjustment mechanism 424) can be coupled to the indicator 416 via a gear reduction mechanism 500. The indicator 416 can be a rotatable indicator that rotates relative to the end cap 490 (for example, see FIG. 21). For example, the end cap 490 can include a reference indicium, such that the rotational position of the indicia on the indicator 416 relative to the end cap 490 can indicate an amount of flex (or radius of curvature) of the shaft 406. In some examples, the end cap 490 can include the indicia and the indicator 416 can include the reference indicum. As such, the reference indicum can be configured to rotate relative to the indicia on the end cap 490 to indicate the amount of flex or radius of curvature of the shaft 406.

Rotation of the knob 411 can drive rotation of both the adjustment mechanism 424 and the indicator 416. When the knob 411 is rotated at a first rate, the gear reduction mechanism 500 can be configured to rotate the indicator 416 at a reduced rate. In this way, the gear reduction mechanism 500 can allow the indicator 416 to make fewer revolutions than the knob 411 as the knob 411 is rotated relative to the handle 404 to adjust the curvature of the shaft 406. As one example, a half revolution of the indicator can indicate the full range of curvature of the shaft 406, even when multiple revolutions of the knob 411 are required to fully flex the shaft 406. This can enable the full range of curvature represented by the indicia on the indicator 416 to be visible to a user of the delivery apparatus 400 from one direction, for example, without having to torque or rotate the handle 404.

The gear reduction mechanism 500 can include one or more pulleys 502 and a belt 504. In some examples, as depicted, the gear reduction mechanism 500 can include two pulleys 502 that extend radially from a sun gear 506. The sun gear 506 can include teeth that are meshed with teeth of gears 440. In this way, rotation of the knob 411 relative to the handle 404 can drive rotation of the sun gear 506 (and therefore pulleys 502) via gears 440.

In some examples, as depicted, the sun gear 506 can be disposed around the distal end portion 488 of the base member 484. In particular, the sun gear 506 can be rotatable relative to the base member 484, which is fixed relative to the handle 404. The teeth of the sun gear 506 can be disposed at a proximal end of the sun gear 506 and the pulleys 502 can extend radially from a distal end of the sun gear 506.

As shown in FIGS. 18-20, the pulleys 502 can be coupled to an inner surface 508 of the belt 504. The outer surface of the belt 504 includes teeth 510. The teeth 510 can be selectively meshed with teeth 496 disposed on an inner surface of the indicator 416. Specifically, the indicator 416 can include a greater number of teeth 496 than the number of teeth 510 of the belt 504.

The pulleys 502 can be configured to push the belt 504 radially outward towards the indicator 416, such that a subset of the teeth 510 of the belt 504 are meshed with a subset of the teeth 496 of the indicator 416 at a given time. Specifically, as the sun gear 506 is rotated via the knob 411, the pulleys 502 are likewise rotated, which selectively pushes the teeth 510 of the belt 504 against the teeth 496 of the indicator 416 to rotate the indicator 416 at a reduced rate. In some instances, for example, only the teeth 496, 510 that are adjacent to the pulleys 502 are meshed and the teeth 496, 510 that are not adjacent to the pulleys 502 are not meshed together. Due to the difference in the number of teeth 496, 510, the gear reduction mechanism 500 is able to rotate the indicator 416 at a slower rate than the knob 411.

In some examples, the gear ratio of the gear reduction mechanism 500 can be altered to allow the indicator 416 to move more or less than half a revolution, for example, as the shaft 406 is moved from an unflexed to a fully flexed configuration. Specifically, the number of teeth 496 of the indicator 416 and/or the number of teeth 510 of the belt 504 can be altered such that the indicator 416 is driven at a different rate that results in a different number of revolutions of the indicator 416 relative to the handle 404, between the unflexed and fully flexed configurations.

Turning to FIGS. 22-24, another exemplary delivery apparatus 600 is shown and described. In some examples, the delivery apparatus 600 can have one or more of the features of the delivery apparatuses 200, 400 discussed above. In some examples, the delivery apparatus can have features that differ from the delivery apparatuses 200, 400.

For example, similar to the delivery apparatus 400, the delivery apparatus 600 can provide for manipulation of a radius of curvature of a shaft of the delivery apparatus 600 independent of an axial displacement of the shaft relative to other components of the delivery apparatus. For example, the shaft of the delivery apparatus 600 can be retracted relative to a prosthetic implant coupled to the delivery apparatus 600 via a shaft displacement mechanism, without adjusting the radius of curvature of the shaft. Similarly, the delivery apparatus 600 can enable the curvature of the shaft to be adjusted via a shaft adjustment mechanism, without changing the axial position of the shaft relative to the prosthetic implant.

Different from the delivery apparatus 400, first and second knobs of the delivery apparatus that control flex and axial displacement of the shaft (discussed below) can be axially offset relative to one another. In other words, the first knob can be disposed at (or closer to) a distal end of a handle and the second knob can be disposed at (or closer to) a proximal end of the handle and a central portion of the handle can be disposed between the first and second knobs. In some examples, the separation of the first and second knobs can allow a housing of the delivery apparatus 600 to have a narrower (lower-profile) distal end portion, which can enable gripping and handling of the delivery apparatus by an operator. Further details of the delivery apparatus 600 are described below.

Similar to delivery apparatuses 200, 400, a prosthetic valve (for example, mechanically-expandable prosthetic valves such as prosthetic valve 100 described herein, self-expandable prosthetic valves, balloon-expandable prosthetic valves, etc.) and/or prosthetic devices other than prosthetic valves, such as stents or grafts can be releasably coupled to the delivery apparatus 600.

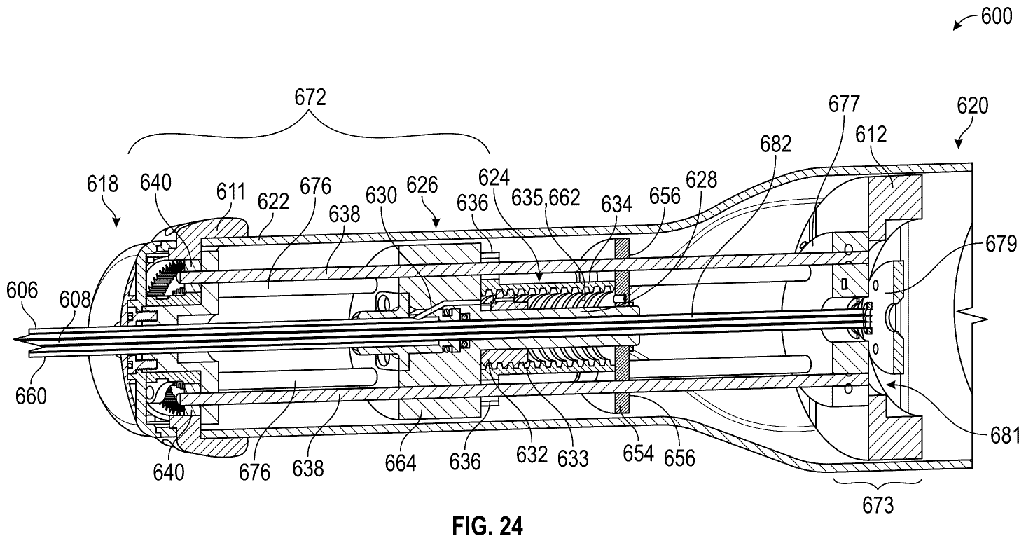

The delivery apparatus 600 in the illustrated example generally includes a handle 604, a first elongated shaft 606 extending distally from the handle 604 and at least one expansion mechanism 608 extending distally through the first shaft 606 (FIG. 24). Although not shown, a prosthetic device, such as a prosthetic heart valve, can be coupled to the expansion mechanism 608. The expansion mechanism 608 can include be one of the expansion mechanisms described herein (for example, any actuator assembly described herein, a balloon for a balloon-expandable prosthetic device, an inner shaft having a self-expandable prosthetic device disposed on an outer surface thereof, etc.) or other types of expansion mechanism suitable for an expandable prosthetic device. In some examples, a distal end portion of the shaft 606 can be sized to house the prosthetic device in its radially compressed, delivery state (for example, as coupled to the expansion mechanism 608) during delivery of the prosthetic valve through the patient's vasculature.

The handle 604 of the delivery apparatus 600 can include one or more control mechanisms (for example, knobs or other actuating mechanisms) for controlling different components of the delivery apparatus 600 in order to expand and/or deploy the prosthetic valve. For example, in the illustrated example the handle 604 includes a first (flex) knob 611 and a second (shaft displacement) knob 612. Although not shown, the handle 604 can include one or more additional knobs, such as an actuation knob similar to the third knob 414 of FIG. 6 or another actuation knob. The first 611 can have one or more of the feature of the first (flex) knob 411, and the second knob 612 can have one or more of the feature of the second (shaft displacement) knob 412.

For example, the first knob 611 can be a rotatable knob configured for advancing the delivery shaft 606 to and/or positioning the delivery shaft 606 at a location at or adjacent a desired implantation location with the patient's body, where rotation of the first knob 611 in a first direction (for example, clockwise) relative to the handle 604 can increase the curvature of the shaft 606 and rotation of the first knob 611 in a second direction (for example, counterclockwise) relative to the handle 604 can decrease the curvature of the shaft 606. In another example, second knob 612 can be a rotatable knob configured to produce axial movement of the first shaft 606 relative to the prosthetic valve in the distal and/or proximal directions in order to deploy the prosthetic valve from the delivery shaft 606 once the prosthetic valve has been advanced to the location at or adjacent the desired implantation location, where rotation of the second knob 612 in a first direction (for example, clockwise) relative to the handle 604 can retract the shaft 606 proximally relative to the prosthetic valve and rotation of the second knob 612 in a second direction (for example, counter-clockwise) relative to the handle 604 can advance the shaft 606 distally.

In some examples, the first knob 611 and/or the second knob 612 can be actuated by sliding the knob, such as by pulling and/or pushing the knob. In some examples, the delivery apparatus 600 can include one or more indicator mechanisms, similar to the indicator 416 discussed above or other indicator mechanisms.

As shown in FIG. 22, the first knob 611 is positioned at a distal end portion 618 of the handle 604. In examples including an indicator mechanism, the indicator mechanism can be proximal or distal relative to the first knob 611. The second knob 612 is axially offset from the first knob 611 in a proximal direction (that is, closer to a proximal end portion 620 of the handle 604). In examples, including a third knob, the third knob can be proximal relative to the second knob 612.

The handle 604 can also include an outer housing 622. As shown in FIG. 22, in some examples, the first knob 611 can be distal relative to the housing 622 and can be uncovered by or exposed from the housing 622. In some examples, the second knob 612 can be a wheel disposed within the housing 622, and the housing 622 can include an opening 613 through which a portion of the second knob 612 extends. In the illustrated example, the second knob 612 can have a smooth surface. In some examples, the second knob 612 can include a coating (for example, a silicone coating) to enable grip of wheel by an operator. In some examples, the second knob 612 can include surface features, such as a textured surface or a surface comprising a plurality of grooves (similar to the first knob 611).

As shown in FIG. 24, the handle 604 can include an adjustment mechanism 624 for adjusting the flex or curvature of the delivery shaft 606 and a displacement mechanism 626 for axially displacing the delivery shaft 606 relative to the expansion mechanisms 608 disposed within the housing 622. In some examples, the adjustment mechanism 624 can be coupled to the displacement mechanism 626. Specifically, the delivery apparatus 600 can include a connector shaft 628 to couple the adjustment mechanism 624 and the displacement mechanism 626.

The adjustment mechanism 624 can be operatively coupled to the first (flex) knob 611. In some examples, the distal end portion of the delivery shaft 606 can be configured to be steerable via the adjustment mechanism 624 based on rotation of the first knob 611 relative to the housing 622. For example, by rotating the knob 611, a curvature of the distal end portion of the delivery shaft 606 can be adjusted so that the distal end portion of the delivery shaft 606 can be oriented in a desired angle. Specifically, to implant a prosthetic device (for example, prosthetic valve 100, prosthetic valve 100, etc.), the distal end portion of the delivery shaft 606 can be steered so that the prosthetic valve can be positioned at a target implantation location.

In addition to the knob 611, the adjustment mechanism 624 (also referred to herein as a “flex assembly”) can include a pull wire 630. The adjustment mechanism 624 can be configured to steer the distal end portion of the delivery shaft 606 via the knob 611 and the pull wire 630 by increasing or decreasing the tension of the pull wire 630. Specifically, a distal end of the pull wire 630 can be connected to the distal end portion of the delivery shaft 606. When the tension of the pull wire 630 changes, the curvature of the distal end portion of the delivery shaft 606 changes in response to the tension of the pull wire 630.

The adjustment mechanism 624 can also include a flex nut 632 and a flex lead member 634 to axially displace the pull wire 630 relative to the handle 604. For example, the pull wire 630 can extend proximally into the handle 604 and a proximal end of the pull wire 630 can be connected to the flex nut 632. In some examples, the proximal end of the pull wire 630 can be connected to the flex nut 632 in a similar manner as the pull wire 430 is connected to the flex nut 432 (that is, via an attachment member (also referred to as a “wire wrap”) for coupling the pull wire 630 to the flex nut 632). In other examples, the proximal end of the pull wire 630 can be connected to the flex nut 632 in a different manner than the pull wire 430 is connected to the flex nut 432 (for example, via fasteners, adhesive, and/or other means of coupling).

In some examples, the delivery apparatus 600 can also include one or more gear assemblies 672 to couple the knob 611 to the other components of the adjustment mechanism 624 disposed within the handle 604. For example, each gear assembly 672 can include one or more first (or proximal) gears 636, one or more rods 638, and one or more second (or distal) gears 640.

As depicted, the gear assemblies 672 can enable the flex nut 632 and the flex lead member 634 of the adjustment mechanism 624 to be disposed proximal (or move proximally) to at least some of the components of the displacement mechanism 626 within the handle 604 (such as a carriage 664 of the displacement mechanism 626). The gear assemblies 672 can enable the adjustment mechanism 624 to be disposed generally proximal to the displacement mechanism 626, even though the knob 611 corresponding to the adjustment mechanism 624 is distal to the knob 612 corresponding to the displacement mechanism 626. For example, the rods 638 can generally extend at least a portion of the length of the handle 604 with the rods 638 extending proximally from the gears 640, through the carriage 664 of the displacement mechanism 626, and at least to the gears 636 which can be coupled to the flex lead member 634 of the adjustment mechanism 624. Different from the delivery apparatus 400, the gear assemblies 672 and the adjustment mechanism 624 can be disposed distally relative to the second (retraction) knob 612 to enable the axial separation of the first knob 611 and the second knob 612.

The flex nut 632 can be disposed around the connector shaft 628, such that the connector shaft 628 extends through an opening of the flex nut 632. The flex nut 632 can be configured to translate axially relative to the connector shaft 628 to adjust the tension of the pull wire 630. In some examples, rotation of the flex lead member 634 can result in the axial translation of the flex nut 632 relative to the connector shaft 628. In some examples, the flex nut 632 can include one or more notches that can engage with guides of the connector shaft 628 and that can be configured to prevent rotational movement of the flex nut 632 relative to the connector shaft 628 during axial translation of the flex nut 632 along the connector shaft 628, similar to the notches 450 and the guides 452 of the delivery apparatus 400.

Further, the connector shaft 628 can include an axially extending slot within a region that extends through the carriage 664 to allow the pull wire 630 to pass from a location that is radially inward of the connector shaft 628 to a location that is radially outward of the connector shaft 628. In some examples, as depicted, the connector shaft 628 includes a central lumen 660 within which the expansion mechanisms 408 are disposed within and extend therethrough.

In some examples, the flex nut 632 includes external threads 633 that can be mated with internal threads 635 of the flex lead member 634. Specifically, the flex lead member 634 can include an inner surface defining a lumen and the internal threads 635, and the external threads 633 of the flex nut 632 can be threadedly engaged with the threads 635 of the lumen's inner surface. In some examples, the flex nut 632 can translate axially within the flex lead member 634 along the threads 635 of the inner surface 662 and along the connector shaft 628, based on rotation of the first knob 611 relative to the handle 604.

In some examples, the connector shaft 628 can be coupled to the adjustment mechanism 624 and the displacement mechanism 626. Specifically, a distal end of the connector shaft 628 can be coupled to a carriage 664 of the displacement mechanism 626. In some examples, as depicted, pins can be used to couple the connector shaft 628 to the carriage 664. In some examples, the connector shaft 628 can be coupled to the carriage 664 in other manners, such as with an adhesive, a friction fit, or other coupling mechanisms. At the proximal end of the connector shaft 628, a flange 654 can be coupled to the rods 638 and the flange 654 can be adjacent to a proximal end of the flex lead member 634. In some examples, the flex lead member 634 can be disposed around the connector shaft 628 and the rods 638 can extend through the openings 656 of the flange 654. The rods 638 can be rotatable relative to the connector shaft 628 within the opening 656. The proximal gears 636 can be disposed on the rods 638 and can be coupled (for example, rotatably coupled) to an outer surface of the flex lead member 634. In some examples, the proximal gears 636 are fixedly coupled to the rods 638.

The flex lead member 634 can include inner threaded surface 662 and an outer surface 668 including outer teeth. As described above, the of the flex nut 632 can include external threads which engage with the internal threads of the flex lead member 634, as shown in FIG. 24. The teeth of the outer surface 668 can be meshed with teeth of the proximal gears 636. As such, when the proximal gears 636 are rotated by rotation of the rods 638, the flex lead member 634 can also be rotated. Due to the threaded connection between the flex lead member 634 and the flex nut 632, the flex nut 632 (and therefore, the pull wire 630) is displaced axially (along the connector shaft 628 and within the flex lead member 634) to change the tension of the pull wire 630 and adjust the curvature or flex of the distal end portion of the delivery shaft 606.

The knob 611 can be coupled to the rods 638 via the distal gears 640. Specifically, the knob 611 can include inner teeth 670 that are meshed with (for example, engaged with) teeth of the distal gears 640 for operatively coupling the knob 611 to the flex lead member 634, such that rotation of the knob 611 drives or results in axial movement of the flex nut 632.

In some examples, knob 611, the distal gears 640, the proximal gears 636, and the rods 638, can have one or more of the features or variations described above with respect to knob 411, the distal gears 440, the proximal gears 436, and the rods 438. For example, the proximal gears 636 and the distal gears 640 can be coupled to the rods 638, such that the gears 636, 640 are not permitted to rotate relative to the rods 638. For example, the rods 638 can be shaped (for example, D-shaped) such that the rods 638 mate with a corresponding opening of the gears 636, 640.

As discussed above, the displacement mechanism 626 can be configured to axially displace delivery shaft 606 relative to the expansion mechanisms 608 (for example, to retract the delivery shaft 606 relative to a prosthetic heart valve coupled to the expansion mechanisms 608). The displacement mechanism 626 can include the second knob 612 and the carriage 664 (also referred to herein as a “displacement nut” or “displacement member”). As described in more detail below, rotation of the knob 612 relative to the handle 604 can drive axial displacement of the carriage 664 (and the shaft 606 coupled thereto) relative to the handle 604 for operation of both of the adjustment mechanism 624 and the displacement mechanism 626.

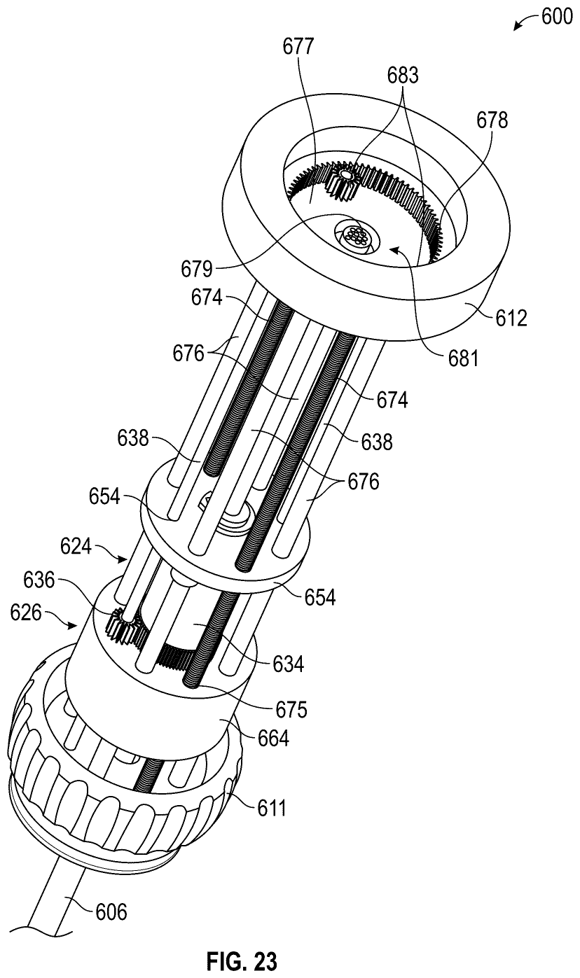

As can be seen in FIGS. 22 and 23, the knob 612 can be a wheel having a portion that is exposed through the opening 613 in the housing 622. In some examples, the exposed portion of the knob 612 is configured to be engaged by a user to rotate the knob 612 relative to the handle 604. Rotation of knob 612 is configured to axially displace one or more components of the shaft displacement mechanism 626 (for example, the displacement nut 664) relative to the knob 612 and the housing 622. As shown in FIG. 24, the displacement nut 664, the connector shaft 628, and one or more components of the adjustment mechanism 624 can be disposed within a central portion of the housing 622 (and the handle 604) that is distal relative to the knob 612.

In some examples, the delivery apparatus 600 can further include one or more gear assemblies 673 to couple the knob 612 to the other components of the displacement mechanism 626 disposed within the handle 604. For example, each gear assembly 673 can include one or more gears 683 and one or more threaded rods 674 (also referred to as “lead screws”). As can be seen in FIG. 23, teeth on an inner surface 678 of the knob 612 can be meshed with (for example, engaged with) the gears 683, which are coupled at proximal ends of the threaded rods 674. In the present example, the delivery apparatus 600 can include two gear assemblies 673 (however only one is shown in FIG. 23) including two gears 683 and two corresponding threaded rods 674 coupled thereto. In other examples, the delivery apparatus 600 can include more or fewer gear assemblies.

In some examples, the threaded rods 674 extend through corresponding axially aligned openings in a disc 677 disposed below the second knob 612, the flange 654, the displacement nut 664 of the displacement mechanism 626, and into the flex knob 611. In some examples, the threaded rods 674 are in threaded engagement with an interior surface of openings 675 within the displacement nut 664, such that rotation of the threaded rods 674 results in axial movement of the displacement nut 664 and retraction of the delivery shaft 606. Specifically, the displacement nut 664 can be coupled to a proximal end portion 682 of the delivery shaft 606 so that axial translation of the displacement nut 664 relative to the expansion mechanisms 608 can cause the delivery shaft 606 to be retracted relative to the expansion mechanisms 608. In this way, rotation of the knob 612 can control axial movement of the delivery shaft 606. In some examples, the proximal end portion 682 of the delivery shaft 606 can be coupled to an attachment member 679 disposed within a central opening 681 defined by the inner surface 678 of the second knob 612.

In some examples, the delivery apparatus 600 can further include support rods 676 (also referred to as “lead rods”) that (similar to the threaded rods 674) extend through corresponding openings in the flange 654, the displacement nut 664 of the displacement mechanism 626, and into the flex knob 611. The support rods 676 can have a smooth outer surface and can provide added support for the displacement nut 664 during axial translation thereof. In the example shown in FIG. 23, the delivery apparatus 600 can include four support rods 676. In other examples, the delivery apparatus 600 can include more support rods 676. In other examples, the delivery apparatus 600 can include fewer support rods 676 or can exclude support rods 676. In some examples, the support rods 676 can be replaced with additional threaded rods 674 coupled to additional gear assemblies 673.

As discussed above, the displacement nut 664 can be axially displaced, for example, as a result of rotation of the knob 612, as the nut 664 translates axially along the rods 638. In addition to the displacement nut 664 being configured to translate axially relative to the rods 638, the rods 638 can be rotatable within the openings of the displacement nut 664, for example, as a result of rotation of the first knob 611.

In some examples, the proximal end portion 682 of the delivery shaft 606 can also be coupled to the connector shaft 628. For example, the proximal end portion 682 of the delivery shaft 606 can be positioned within the lumen 660 of the connector shaft 628 and coupled thereto (for example, with an adhesive, etc.). In such examples, the connector shaft 628 can be coupled to the displacement nut 664 (for example, via pins, fasteners, and/or an adhesive). In this way, the relative axial positioning of the delivery shaft 606, the displacement nut 664, and the connector shaft 628 can be fixed such that axial displacement of one of these components results in axial displacement of the others.

In some examples, the delivery apparatus 600 can further include an indicator mechanism coupled to the first knob 611, such as, for example, an indicator similar to the indicator 416 or another indicator.

In some examples, operation of the adjustment mechanism 624 for adjusting the flex or curvature of the delivery shaft 606 can be similar to the operations described above with respect to the adjustment mechanism 424. In some examples, operation of the displacement mechanism 626 for axially displacing the delivery shaft 606 relative to the expansion mechanisms 608 disposed within the housing 622 can be similar to the operations described above with respect to the displacement mechanism 426. In other examples, operations of the adjustment mechanism 624 and/or the displacement mechanism 626 can differ from the those of the adjustment mechanism 424 and the displacement mechanism 426.

As discussed above, to adjust the flex or curvature of the distal end of the delivery shaft 606, a user can engage and rotate the knob 611 relative to handle 604. As the teeth on the inner surface of the knob 611 are meshed with the distal gears 640, rotation of the knob 611 can drive rotation of the gears 640 and the rods 638 coupled thereto. Further, rotation of the rods 638 results in rotation of the proximal gears 636 which meshed with teeth on the outer surface of the flex lead member 634, and thereby drives rotation of the flex lead member 634. Due to the threaded connection between the flex lead member 634 and the flex nut 632, the flex nut 632 (and therefore, the pull wire 630) is displaced axially (along the connector shaft 628 and within the flex lead member 634) to change the tension of the pull wire 630 and adjust the curvature or flex of the distal end portion of the delivery shaft 606, without retracting the delivery shaft 606 relative to the expansion mechanisms 608 and/or a prosthetic valve coupled at the distal end of the delivery shaft.

In some examples, to adjust the axial positioning of the distal end of the delivery shaft 606 relative to the expansion mechanisms 608 and a prosthetic valve coupled thereto, the knob 612 can be rotated relative to handle 604. For example, a user can engage the exposed portion of the knob 612 to rotate the knob. Upon rotation of the knob 612 in a first direction, the gear assemblies 673 drive rotation of the threaded rods 674, which threadedly engage the threaded interior surfaces of the openings 675 in the displacement nut 664. The threaded engagement and rotation of the threaded rods 674 can drive the displacement nut 464 in an axial direction such that the displacement nut 664 is translated (for example, proximally) along the rods 638. Due to the proximal end portion 682 of the delivery shaft 606 being fixedly coupled to the displacement nut 664, the shaft 606 is also translated axially (for example, proximally), relative to the prosthetic valve and the expansion mechanisms 608, which can enable the prosthetic valve (and in some examples, the expansion mechanisms 608) to be unsheathed from the distal end of the shaft 606, for example, to enable radial expansion of the prosthetic valve coupled to the expansion mechanisms 608.

Further, due to the connection between the displacement nut 664 and the connector shaft 628, the connector shaft 628 and components of the adjustment mechanism 624 are also driven axially by the displacement nut 664. For example, rotation of the second knob 612 in the first direction can result in axial displacement (for example, proximal displacement) of the connector shaft 628, the pull wire 630, the flex nut 632, the flex lead member 634, and the proximal gears 636. As such, in some examples, when the knob 612 is rotated, the shaft 606 and the pull wire 630 (which is coupled to the distal end of the shaft 606) are both axially displaced by the same amount. This enables the axial position of the shaft 606 to be adjusted relative to the expansion mechanisms 608 (and the prosthetic valve) without changing the tension of the pull wire 630 and/or the radius of curvature of the distal end of the shaft 606. This can, for example, allow the prosthetic valve to be deployed from the distal end of the shaft 606 without changing the curvature of the shaft 606. As such, the delivery apparatus 600 can, among other things, improve the process of implanting a prosthetic device and/or reduce the time of the implantation procedure.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat, radiation, and/or chemicals, etc.) to ensure they are safe for use with patients, and any of the methods herein can include sterilization of the associated system, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A delivery apparatus for a prosthetic valve, the delivery apparatus comprising: a handle body; a shaft displacement mechanism coupled to the handle body, the shaft displacement mechanism configured to axially displace a shaft relative to the handle body; a shaft adjustment mechanism coupled to the handle body, the adjustment mechanism configured to adjust a curvature of the shaft, the shaft adjustment mechanism comprising a pull wire coupled to a distal end of the shaft; a first knob operatively coupled to the shaft displacement mechanism and rotatable relative to the handle body, wherein rotating the first knob relative to the handle body simultaneously axially displaces the shaft and the pull wire relative to the handle body; and a second knob operatively coupled to the shaft adjustment mechanism and rotatable relative to the handle body, wherein rotating the second knob relative to the handle body adjusts a radius of the curvature of the shaft independent of an axial displacement of the shaft.

Example 2. The delivery apparatus of any example herein, particularly example 1, wherein rotating the first knob relative to the handle body in a first direction displaces the shaft and the pull wire in a proximal direction relative to the handle body.

Example 3. The delivery apparatus of any example herein, particularly either example 1 or example 2, wherein rotating the first knob relative to the handle body in a second direction displaces the shaft and the pull wire in a distal direction relative to the handle body.

Example 4. The delivery apparatus of any example herein, particularly any one of examples 1-3, wherein rotating the second knob relative to the handle body in a first direction increases the radius of curvature of the shaft.

Example 5. The delivery apparatus of any example herein, particularly any one of examples 1-4, wherein rotating the second knob relative to the handle body in a second direction decreases the radius of curvature of the shaft.

Example 6. The delivery apparatus of any example herein, particularly any one of examples 1-5, wherein the shaft adjustment mechanism further comprises: a rotatable, adjustment barrel having a lumen that includes a threaded inner surface; and an adjustment nut coupled to the pull wire and disposed within the lumen, wherein the adjustment nut includes a threaded outer surface coupled to the threaded inner surface of the adjustment barrel, wherein the adjustment nut is configured to translate axially relative to the adjustment barrel in response to rotation of the adjustment barrel.

Example 7. The delivery apparatus of any example herein, particularly example 6, wherein a proximal end of the pull wire is coupled to the adjustment nut.

Example 8. The delivery apparatus of any example herein, particularly example 7, wherein the adjustment nut includes an attachment member extending radially from a main body of the adjustment nut, wherein the proximal end of the pull wire is wrapped around the attachment member.

Example 9. The delivery apparatus of any example herein, particularly any one of examples 6-8, further comprising a connector shaft coupled to the adjustment mechanism and the shaft displacement mechanism.

Example 10. The delivery apparatus of any example herein, particularly example 9, wherein the adjustment nut is disposed circumferentially around the connector shaft and movable axially relative to the connector shaft.

Example 11. The delivery apparatus of any example herein, particularly example 10, wherein the connector shaft comprises a guide projection extending along an axial length of the connector shaft, wherein the adjustment nut comprises a notch aligned with the guide projection, wherein axial displacement of the adjustment nut relative to the connector shaft causes the notch to move along the guide projection.

Example 12. The delivery apparatus of any example herein, particularly any one of examples 9-11, further comprising a gear system operatively coupling the shaft adjustment mechanism and the second knob.

Example 13. The delivery apparatus of any example herein, particularly example 12, wherein the gear system comprises: at least one distal gear having teeth meshed with inner teeth of the second knob; at least one proximal gear having teeth meshed with outer teeth of the adjustment barrel; and at least one rod, wherein the at least one distal gear and the at least one proximal gear are coupled to the at least one rod.

Example 14. The delivery apparatus of any example herein, particularly any one of examples 1-13, further comprising a rotatable indicator coupled to the shaft adjustment mechanism and configured to indicate the curvature of the shaft upon rotation of the second knob.

Example 15. The delivery apparatus of any example herein, particularly example 14, further comprising a gear reduction mechanism operatively coupled between the shaft adjustment mechanism and the indicator, wherein rotation of the second knob relative to the handle body results in rotation of the indicator at a reduced rate based on the gear reduction mechanism.

Example 16. The delivery apparatus of any example herein, particularly example 15, wherein the gear reduction mechanism is a harmonic drive comprising one or more pulleys and a belt.

Example 17. The delivery apparatus of any example herein, particularly any one of examples 1-16, further comprising the shaft, wherein the shaft is configured to encapsulate a prosthetic implant.

Example 18. The delivery apparatus of any example herein, particularly example 17, wherein the shaft displacement mechanism comprises a displacement nut coupled to the shaft, wherein the displacement nut is threadedly coupled to the first knob, wherein rotation of the first knob relative to the handle body results in axial displacement of the displacement nut and the shaft relative to the handle body.

Example 19. The delivery apparatus of any example herein, particularly either example 17 or example 18, wherein the prosthetic implant comprises one of: a prosthetic heart valve or a stent.

Example 20. The delivery apparatus of any example herein, particularly any one of examples 17-19, wherein the prosthetic implant is self-expandable, balloon-expandable, and/or mechanically-expandable.

Example 21. The delivery apparatus of any example herein, particularly any one of examples 17-20, further comprising an expansion mechanism disposed within the shaft and coupled to the prosthetic implant, wherein the expansion mechanism comprises one of: at least one actuator assembly for mechanical expansion of the prosthetic implant, an inflatable balloon catheter for balloon expansion of the prosthetic implant, and an inner shaft for self-expansion of the prosthetic implant.

Example 22. A handle for a delivery apparatus for a prosthetic valve, the handle comprising: a handle body; a shaft displacement mechanism coupled to the handle body, the shaft displacement mechanism configured to axially displace a shaft relative to the handle body; a shaft adjustment mechanism coupled to the handle body, the adjustment mechanism configured to adjust a curvature of the shaft, the shaft adjustment mechanism comprising a pull wire coupled to a distal end of the shaft; a first knob operatively coupled to the shaft displacement mechanism and rotatable relative to the handle body, wherein rotating the first knob relative to the handle body simultaneously axially displaces the shaft and the pull wire relative to the handle body; and a second knob operatively coupled to the shaft adjustment mechanism and rotatable relative to the handle body, wherein rotating the second knob relative to the handle body adjusts the curvature of the shaft independent of an axial displacement of the shaft.

Example 23. The handle of any example herein, particularly example 22, wherein rotating the first knob relative to the handle body in a first direction displaces the shaft and the pull wire in a proximal direction relative to the handle body.

Example 24. The handle of any example herein, particularly either example 22 or example 23, wherein rotating the first knob relative to the handle body in a second direction displaces the shaft and the pull wire in a distal direction relative to the handle body.

Example 25. The handle of any example herein, particularly any one of examples 22-24, wherein rotating the second knob relative to the handle body in a first direction increases the radius of curvature of the shaft.

Example 26. The handle of any example herein, particularly any one of examples 22-25, wherein rotating the second knob relative to the handle body in a second direction decreases the radius of curvature of the shaft.

Example 27. The handle of any example herein, particularly any one of examples 22-26, wherein the shaft adjustment mechanism comprises: a rotatable, adjustment barrel having a lumen that includes a threaded inner surface; and an adjustment nut coupled to the pull wire and disposed within the lumen, wherein the adjustment nut includes a threaded outer surface coupled to the threaded inner surface of the adjustment barrel, wherein the adjustment nut is configured to translate axially relative to the adjustment barrel in response to rotation of the adjustment barrel.

Example 28. The handle of any example herein, particularly example 27, wherein a proximal end of the pull wire is coupled to the adjustment nut.

Example 29. The handle of any example herein, particularly example 28, wherein the adjustment nut includes an attachment member extending radially from a main body of the adjustment nut, wherein the proximal end of the pull wire is wrapped around the attachment member.

Example 30. The handle of any example herein, particularly any one of examples 27-29, further comprising a connector shaft coupled to the adjustment mechanism and the shaft displacement mechanism.

Example 31. The handle of any example herein, particularly example 30, wherein the adjustment nut is disposed circumferentially around the connector shaft and movable axially relative to the connector shaft.

Example 32. The handle of any example herein, particularly example 31, wherein the connector shaft comprises a guide projection extending along an axial length of the connector shaft, wherein the adjustment nut comprises a notch aligned with the guide projection, wherein axial displacement of the adjustment nut relative to the connector shaft causes the notch to move along the guide projection.

Example 33. The handle of any example herein, particularly any one of examples 30-32, further comprising a gear system operatively coupling the shaft adjustment mechanism and the second knob.

Example 34. The handle of any example herein, particularly example 33, wherein the gear system comprises: at least one distal gear having teeth meshed with inner teeth of the second knob; at least one proximal gear having teeth meshed with outer teeth of the adjustment barrel; and at least one rod, wherein the at least one distal gear and the at least one proximal gear are coupled to the at least one rod.

Example 35. The handle of any example herein, particularly any one of examples 22-34, further comprising a rotatable indicator coupled to the shaft adjustment mechanism and configured to indicate the curvature of the shaft upon rotation of the second knob.

Example 36. The handle of any example herein, particularly example 35, further comprising a gear reduction mechanism operatively coupled between the shaft adjustment mechanism and the indicator, wherein rotation of the second knob relative to the handle body results in rotation of the indicator at a reduced rate based on the gear reduction mechanism.

Example 37. The handle of any example herein, particularly example 36, wherein the gear reduction mechanism is a harmonic drive comprising one or more pulleys and a belt.

Example 38. A delivery apparatus for a prosthetic valve, the delivery apparatus comprising: a delivery shaft; an expansion mechanism disposed within the delivery shaft; a displacement nut coupled to a proximal end portion of the delivery shaft, the displacement nut configured to axially displace the delivery shaft relative to the expansion mechanism; a shaft adjustment mechanism comprising a pull wire coupled to a distal end portion of the delivery shaft, the adjustment mechanism configured to adjust a curvature of the delivery shaft; a connector shaft coupled to the displacement nut and the shaft adjustment mechanism; a first knob operatively coupled to the displacement nut and rotatable relative to the expansion mechanism, wherein rotating the first knob relative to the expansion mechanism simultaneously axially displaces the displacement nut, the delivery shaft, the pull wire, and the connector shaft relative to the expansion mechanism; and a second knob operatively coupled to the shaft adjustment mechanism and rotatable relative to the expansion mechanism, wherein rotating the second knob relative to the expansion mechanism adjusts a tension of the pull wire independent of an axial displacement of the delivery shaft.

Example 39. The delivery apparatus of any example herein, particularly example 38, wherein rotating the first knob relative to the expansion mechanism in a first direction displaces the displacement nut, the delivery shaft, the pull wire, and the connector shaft in a proximal direction relative to the expansion mechanism.

Example 40. The delivery apparatus of any example herein, particularly either example 38 or example 39, wherein rotating the first knob relative to the expansion mechanism in a second direction displaces the displacement nut, the delivery shaft, the pull wire, and the connector shaft in a distal direction relative to the expansion mechanism.

Example 41. The delivery apparatus of any example herein, particularly any one of examples 38-40, wherein rotating the second knob relative to the expansion mechanism in a first direction increases the tension of the pull wire.

Example 42. The delivery apparatus of any example herein, particularly any one of examples 38-41, wherein rotating the second knob relative to the expansion mechanism in a second direction decreases the tension of the pull wire.

Example 43. The delivery apparatus of any example herein, particularly any one of examples 38-42, wherein a proximal end of the connector shaft includes a flange, and wherein a distal end of the connector shaft is coupled to the displacement nut.

Example 44. The delivery apparatus of any example herein, particularly example 43, wherein the shaft adjustment mechanism further comprises: a rotatable adjustment barrel having a lumen that includes a threaded inner surface; and an adjustment nut coupled to the pull wire and disposed within the lumen, wherein the adjustment nut includes a threaded outer surface coupled to the threaded inner surface of the adjustment barrel, wherein the adjustment nut is configured to translate axially relative to the adjustment barrel in response to rotation of the adjustment barrel.

Example 45. The delivery apparatus of any example herein, particularly example 44, wherein the adjustment barrel is disposed circumferentially around the connector shaft and axially between the displacement nut and the flange.

Example 46. The delivery apparatus of any example herein, particularly either example 44 or example 45, wherein the displacement nut is distal to the adjustment barrel and the adjustment nut.

Example 47. The delivery apparatus of any example herein, particularly any one of examples 44-46, further comprising a gear system operatively coupling the shaft adjustment mechanism and the second knob.

Example 48. The delivery apparatus of any example herein, particularly example 47, wherein the gear system comprises: at least one distal gear having teeth meshed with inner teeth of the second knob; at least one proximal gear having teeth meshed with outer teeth of the adjustment barrel; and at least one rod, wherein the at least one distal gear and the at least one proximal gear are coupled to the at least one rod.

Example 49. The delivery apparatus of any example herein, particularly example 48, wherein the at least one rod extends through at least one opening of the displacement nut and at least one opening of the flange.

Example 50. The delivery apparatus of any example herein, particularly either example 48 or example 49, wherein the at least one proximal gear extends an axial length of the adjustment barrel.

Example 51. The delivery apparatus of any example herein, particularly either example 48 or example 49, wherein an axial length of the at least one proximal gear is less than an axial length of the adjustment barrel.

Example 52. The delivery apparatus of any example herein, particularly example 51, further comprising a spacer coupled to the at least one rod, wherein the spacer is distal to the flange and proximal to the displacement nut.

Example 53. The delivery apparatus of any example herein, particularly any one of examples 38-52, further comprising a rotatable indicator coupled to the shaft adjustment mechanism and configured to indicate a curvature of the delivery shaft upon rotation of the second knob.

Example 54. The delivery apparatus of any example herein, particularly example 53, further comprising a gear reduction mechanism operatively coupled between the shaft adjustment mechanism and the indicator, wherein rotation of the second knob relative to the expansion mechanism results in rotation of the indicator at a reduced rate based on the gear reduction mechanism.

Example 55. The delivery apparatus of any example herein, particularly example 54, wherein the gear reduction mechanism is a harmonic drive comprising one or more pulleys and a belt.

Example 56. The delivery apparatus of any example herein, particularly any one of examples 38-55, wherein the distal end portion of the delivery shaft is coupled to the connector shaft.

Example 57. The delivery apparatus of any example herein, particularly any one of examples 38-56, wherein the connector shaft is coupled to the displacement nut via one or more pins.

Example 58. The delivery apparatus of any example herein, particularly any one of examples 38-57, wherein the first knob is proximal to the second knob.

Example 59. The delivery apparatus of any example herein, particularly any one of examples 38-58, wherein the adjustment nut includes an attachment member extending radially from a main body of the adjustment nut, wherein a proximal end of the pull wire is wrapped around the attachment member.

Example 60. The delivery apparatus of any example herein, particularly any one of examples 38-59, wherein the adjustment nut is disposed circumferentially around the connector shaft and movable axially relative to the connector shaft.

Example 61. The delivery apparatus of any example herein, particularly example 60, wherein the connector shaft comprises a guide projection extending along an axial length of the connector shaft, wherein the adjustment nut comprises a notch aligned with the guide projection, wherein axial displacement of the adjustment nut relative to the connector shaft results in displacement of the notch along the guide projection.

Example 62. The delivery apparatus of any example herein, particularly any one of examples 38-61, wherein the delivery shaft is configured to encapsulate a prosthetic implant.

Example 63. The delivery apparatus of any example herein, particularly example 62, wherein the prosthetic implant comprises one of: a prosthetic heart valve or a stent.

Example 64. The delivery apparatus of any example herein, particularly either example 62 or example 63, wherein the prosthetic implant is self-expandable, balloon-expandable, and/or mechanically-expandable.

Example 65. The delivery apparatus of any example herein, particularly any one of examples 62-64, wherein the expansion mechanism comprises one of: at least one actuator assembly for mechanical expansion of the prosthetic implant, an inflatable balloon catheter for balloon expansion of the prosthetic implant, and an inner shaft for self-expansion of the prosthetic implant.

Example 66. A delivery apparatus for a prosthetic valve, the delivery apparatus comprising: a delivery shaft; at least one expansion mechanism disposed within the delivery shaft; a displacement member coupled to a proximal end portion of the delivery shaft, the displacement member configured to axially displace the delivery shaft relative to the expansion mechanism; a pull wire coupled to a distal end of the delivery shaft, the pull wire configured to adjust a curvature of the delivery shaft; an adjustment nut coupled to the pull wire, wherein the adjustment nut includes a threaded outer surface; and a rotatable, adjustment barrel having a threaded inner surface coupled to the threaded outer surface of the adjustment nut, wherein rotation of the adjustment barrel relative to the expansion member results in axial displacement of the adjustment nut relative to the adjustment barrel.

Example 67. The delivery apparatus of any example herein, particularly example 66, further comprising a first knob operatively coupled to the displacement member and rotatable relative to the expansion mechanism, wherein rotating the first knob relative to the expansion mechanism simultaneously axially displaces the displacement member and at least one of: the delivery shaft, the pull wire, the adjustment nut, and the adjustment barrel, relative to the expansion mechanism.

Example 68. The delivery apparatus of any example herein, particularly example 67, wherein rotating the first knob relative to the expansion mechanism simultaneously axially displaces the displacement member, the delivery shaft, the pull wire, the adjustment nut, and the adjustment barrel relative to the expansion mechanism.

Example 69. The delivery apparatus of any example herein, particularly either example 67 or example 68, wherein rotating the first knob relative to the expansion mechanism in a first direction displaces the displacement member in a proximal direction relative to the expansion mechanism.

Example 70. The delivery apparatus of any example herein, particularly any one of examples 67-69, wherein rotating the first knob relative to the expansion mechanism in a second direction displaces the displacement member in a distal direction relative to the expansion mechanism.

Example 71. The delivery apparatus of any example herein, particularly any one of examples 66-70, further comprising a second knob operatively coupled to the adjustment barrel and rotatable relative to the expansion mechanism, wherein rotating the second knob relative to the expansion mechanism results in rotation of the adjustment barrel.

Example 72. The delivery apparatus of any example herein, particularly example 71, wherein rotation of the adjustment barrel results in an adjustment of a tension of the pull wire independent of an axial displacement of the delivery shaft.

Example 73. The delivery apparatus of any example herein, particularly either example 71 or example 72, wherein rotating the second knob relative to the expansion mechanism in a first direction increases the tension of the pull wire.

Example 74. The delivery apparatus of any example herein, particularly any one of examples 71-73, wherein rotating the second knob relative to the expansion mechanism in a second direction decreases the tension of the pull wire.

Example 75. The delivery apparatus of any example herein, particularly any one of examples 71-74, further comprising a gear system operatively coupling the adjustment barrel and the second knob.

Example 76. The delivery apparatus of any example herein, particularly example 75, wherein the gear system comprises: at least one distal gear having teeth meshed with inner teeth of the second knob; at least one proximal gear having teeth meshed with outer teeth of the adjustment barrel; and at least one rod, wherein the at least one distal gear and the at least one proximal gear are coupled to the at least one rod.

Example 77. The delivery apparatus of any example herein, particularly example 76, wherein the at least one proximal gear extends an axial length of the adjustment barrel.

Example 78. The delivery apparatus of any example herein, particularly example 76, wherein an axial length of the at least one proximal gear is less than an axial length of the adjustment barrel.

Example 79. The delivery apparatus of any example herein, particularly any one of examples 66-78, wherein the displacement member is distal to the adjustment barrel and the adjustment nut.

Example 80. The delivery apparatus of any example herein, particularly any one of examples 66-79, further comprising a connector shaft coupled to the displacement member and the shaft adjustment mechanism.

Example 81. The delivery apparatus of any example herein, particularly example 80, wherein a proximal end of the connector shaft includes a flange, and wherein a distal end of the connector shaft is coupled to the displacement member.

Example 82. The delivery apparatus of any example herein, particularly example 81, wherein the adjustment barrel is disposed circumferentially around the connector shaft and axially between the displacement member and the flange.

Example 83. The delivery apparatus of any example herein, particularly any one of examples 80-82, wherein the adjustment member is disposed circumferentially around the connector shaft and movable axially relative to the connector shaft.

Example 84. The delivery apparatus of any example herein, particularly example 83, wherein the connector shaft comprises a guide projection extending along an axial length of the connector shaft, wherein the adjustment member comprises a notch aligned with the guide projection, wherein axial displacement of the adjustment member relative to the connector shaft results in displacement of the notch along the guide projection.

Example 85. The delivery apparatus of any example herein, particularly any one of examples 66-84, further comprising a rotatable indicator operatively coupled to the second knob and configured to indicate a curvature of the delivery shaft upon rotation of the second knob.

Example 86. The delivery apparatus of any example herein, particularly example 85, further comprising a gear reduction mechanism operatively coupled between the second knob and the indicator, wherein rotation of the second knob relative to the expansion mechanism results in rotation of the indicator at a reduced rate based on the gear reduction mechanism.

Example 87. The delivery apparatus of any example herein, particularly example 86, wherein the gear reduction mechanism is a harmonic drive comprising one or more pulleys and a belt.

Example 88. The delivery apparatus of any example herein, particularly any one of examples 66-87, wherein the distal end portion of the delivery shaft is coupled to the connector shaft.

Example 89. The delivery apparatus of any example herein, particularly any one of examples 66-88, wherein the connector shaft is coupled to the displacement member via one or more pins.

Example 90. The delivery apparatus of any example herein, particularly any one of examples 66-89, wherein the first knob is proximal to the second knob.

Example 91. The delivery apparatus of any example herein, particularly any one of examples 66-90, wherein the adjustment member includes a radial projection, wherein a proximal end of the pull wire is wrapped around the radial projection.

Example 92. The delivery apparatus of any example herein, particularly any one of examples 66-91, wherein the delivery shaft is configured to encapsulate a prosthetic implant.

Example 93. The delivery apparatus of any example herein, particularly example 92, wherein the prosthetic implant comprises one of: a prosthetic heart valve or a stent.

Example 94. The delivery apparatus of any example herein, particularly either example 92 or example 93, wherein the prosthetic implant is self-expandable, balloon-expandable, and/or mechanically-expandable.

Example 95. The delivery apparatus of any example herein, particularly any one of examples 92-94, wherein the expansion mechanism comprises one of: at least one actuator assembly for mechanical expansion of the prosthetic implant, an inflatable balloon catheter for balloon expansion of the prosthetic implant, and an inner shaft for self-expansion of the prosthetic implant.

Example 96. A method comprising: sterilizing the delivery apparatus of any one of examples 1-21.

Example 97. A method comprising: sterilizing the handle of any one of examples 22-37.

Example 98. A method comprising: sterilizing a delivery apparatus comprising the handle of any one of examples 22-37 and a shaft coupled to the handle.

Example 99. A method comprising: sterilizing the delivery apparatus of any one of examples 38-95.

Example 100. A method of implanting a prosthetic implant, the method comprising: adjusting a curvature of a delivery shaft that retains the prosthetic implant relative to a longitudinal axis of a handle coupled to the delivery shaft; and displacing the delivery shaft relative to the prosthetic implant, wherein the curvature is maintained during displacement.

Example 101. The method of any example herein, particularly example 100, wherein adjusting the curvature comprises rotating a first knob relative to the handle.

Example 102. The method of any example herein, particularly either example 100 or example 101, wherein displacing the delivery shaft relative to the prosthetic implant comprises rotating a second knob relative to the handle.

Example 103. The delivery apparatus of any example disclosed herein, particularly any one of examples 1-17, wherein the shaft displacement mechanism comprises a displacement nut coupled to the shaft, wherein the displacement nut is threadedly coupled to one or more threaded rods, each of the one or more threaded rods coupled to a gear meshed with an interior surface of the first knob, wherein rotation of the first knob relative to the handle body results in axial displacement of the displacement nut and the shaft relative to the handle body.

Example 104. The delivery apparatus of any example disclosed herein, particularly example 103, wherein the first knob is proximal of the second knob and the displacement nut, and wherein the first knob and the second knob are axially separated on the handle body.

Example 105. The delivery apparatus of any example disclosed herein, particularly any one of examples 38-65, wherein the displacement nut is threadedly coupled to one or more threaded rods, each of the one or more threaded rods coupled to a gear meshed with an interior surface of the first knob, wherein rotation of the first knob results in axial movement of the displacement nut and the shaft.

Example 106. The delivery apparatus of any example disclosed herein, particularly example 105, wherein the first knob is proximal of the second knob and the displacement nut, and wherein the first knob and the second knob are axially separated on a handle of the delivery apparatus.

The features described herein with regard to any example can be combined with other features described in any one or more of the other examples, unless otherwise stated. For example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A delivery apparatus for a prosthetic valve, the delivery apparatus comprising:
a handle body;
a shaft displacement mechanism coupled to the handle body, the shaft displacement mechanism configured to axially displace a shaft relative to the handle body;
a shaft adjustment mechanism coupled to the handle body, the shaft adjustment mechanism configured to adjust a curvature of the shaft, the shaft adjustment mechanism comprising a pull wire coupled to a distal end of the shaft;
a first knob operatively coupled to the shaft displacement mechanism and rotatable relative to the handle body, wherein rotating the first knob relative to the handle body simultaneously axially displaces the shaft and the pull wire relative to the handle body; and
a second knob operatively coupled to the shaft adjustment mechanism and rotatable relative to the handle body, wherein rotating the second knob relative to the handle body adjusts a radius of the curvature of the shaft independent of an axial displacement of the shaft.

2. The delivery apparatus of claim 1, wherein rotating the second knob relative to the handle body in a first direction increases the radius of curvature of the shaft.

3. The delivery apparatus of claim 1, wherein rotating the second knob relative to the handle body in a second direction decreases the radius of curvature of the shaft.

4. The delivery apparatus of claim 1, wherein the shaft adjustment mechanism further comprises:
a rotatable, adjustment barrel having a lumen that includes a threaded inner surface; and
an adjustment nut coupled to the pull wire and disposed within the lumen, wherein the adjustment nut includes a threaded outer surface coupled to the threaded inner surface of the adjustment barrel, wherein the adjustment nut is configured to translate axially relative to the adjustment barrel in response to rotation of the adjustment barrel.

5. The delivery apparatus of claim 4, wherein a proximal end of the pull wire is coupled to the adjustment nut.

6. The delivery apparatus of claim 5, wherein the adjustment nut includes an attachment member extending radially from a main body of the adjustment nut, wherein the proximal end of the pull wire is wrapped around the attachment member.

7. The delivery apparatus of claim 1, further comprising a rotatable indicator coupled to the shaft adjustment mechanism and configured to indicate the curvature of the shaft upon rotation of the second knob.

8. The delivery apparatus of claim 7, further comprising a gear reduction mechanism operatively coupled between the shaft adjustment mechanism and the indicator, wherein rotation of the second knob relative to the handle body results in rotation of the indicator at a reduced rate based on the gear reduction mechanism.

9. The delivery apparatus of claim 1, wherein the shaft displacement mechanism comprises a displacement nut coupled to the shaft, wherein the displacement nut is threadedly coupled to one or more threaded rods, each of the one or more threaded rods coupled to a gear meshed with an interior surface of the first knob, wherein rotation of the first knob relative to the handle body results in axial displacement of the displacement nut and the shaft relative to the handle body.

10. The delivery apparatus of claim 9, wherein the first knob is proximal of the second knob and the displacement nut, and wherein the first knob and the second knob are axially separated on the handle body.

11. A delivery apparatus for a prosthetic valve, the delivery apparatus comprising:
a delivery shaft;
an expansion mechanism disposed within the delivery shaft;
a displacement nut coupled to a proximal end portion of the delivery shaft, the displacement nut configured to axially displace the delivery shaft relative to the expansion mechanism;
a shaft adjustment mechanism comprising a pull wire coupled to a distal end portion of the delivery shaft, the shaft adjustment mechanism configured to adjust a curvature of the delivery shaft;
a connector shaft coupled to the displacement nut and the shaft adjustment mechanism;
a first knob operatively coupled to the displacement nut and rotatable relative to the expansion mechanism, wherein rotating the first knob relative to the expansion mechanism simultaneously axially displaces the displacement nut, the delivery shaft, the pull wire, and the connector shaft relative to the expansion mechanism; and
a second knob operatively coupled to the shaft adjustment mechanism and rotatable relative to the expansion mechanism, wherein rotating the second knob relative to the expansion mechanism adjusts a tension of the pull wire independent of an axial displacement of the delivery shaft.

12. The delivery apparatus of claim 11, wherein rotating the second knob relative to the expansion mechanism in a first direction increases the tension of the pull wire.

13. The delivery apparatus of claim 11, wherein rotating the second knob relative to the expansion mechanism in a second direction decreases the tension of the pull wire.

14. The delivery apparatus of claim 11, wherein a proximal end of the connector shaft includes a flange, and wherein a distal end of the connector shaft is coupled to the displacement nut.

15. The delivery apparatus of claim 14, wherein the shaft adjustment mechanism further comprises:
a rotatable adjustment barrel having a lumen that includes a threaded inner surface;
and
an adjustment nut coupled to the pull wire and disposed within the lumen, wherein the adjustment nut includes a threaded outer surface coupled to the threaded inner surface of the adjustment barrel, wherein the adjustment nut is configured to translate axially relative to the adjustment barrel in response to rotation of the adjustment barrel.

16. The delivery apparatus of claim 15, further comprising a gear system operatively coupling the shaft adjustment mechanism and the second knob.

17. The delivery apparatus of claim 16, wherein the gear system comprises:
at least one distal gear having teeth meshed with inner teeth of the second knob;
at least one proximal gear having teeth meshed with outer teeth of the adjustment barrel; and
at least one rod, wherein the at least one distal gear and the at least one proximal gear are coupled to the at least one rod.

18. The delivery apparatus of claim 17, wherein the at least one rod extends through at least one opening of the displacement nut and at least one opening of the flange.

19. The delivery apparatus of claim 11, wherein the displacement nut is threadedly coupled to one or more threaded rods, each of the one or more threaded rods coupled to a gear meshed with an interior surface of the first knob, wherein rotation of the first knob results in axial movement of the displacement nut and the delivery shaft.

20. The delivery apparatus of claim 19, wherein the first knob is proximal of the second knob and the displacement nut, and wherein the first knob and the second knob are axially separated on a handle of the delivery apparatus.
